(19) United States
(12) Patent Application Publication
(10) Pub. No.: US 2012/0288866 A1
(43) Pub. Date: Nov. 15, 2012

(54) SYSTEMS AND METHODS FOR PRODUCING AN EVAPORATION BARRIER IN A REACTION CHAMBER

(75) Inventors: Lynn KOZMA, Sunol, CA (US); Maureen FAIRCHILD, Alameda, CA (US); Jesus CHING, Saratoga, CA (US)

(73) Assignee: GenturaDx, Inc., Tortola, VG (unknown)

(21) Appl. No.: 13/469,714

(22) Filed: May 11, 2012

Related U.S. Application Data
(60) Provisional application No. 61/484,787, filed on May 11, 2011.

(51) Int. Cl. C12Q 1/68; C12P 19/34; C12M 1/00

(52) U.S. Cl. 435/6.12; 435/289.1; 435/91.2

(57) ABSTRACT

The embodiments described herein relate to systems and methods for producing an evaporation barrier in a PCR vial. In some embodiments, beads with a particular distribution in diameters can be used to produce a barrier for reducing the evaporation of liquid PCR samples within the PCR vial. In some embodiments, the beads can be pre-filled in the PCR vial. In use, liquid samples and/or liquid reagents can be introduced in the PCR vial pre-filled with the beads, such that the beads can be driven to the surface of the liquid PCR sample through the buoyancy of the beads.

### CROSS-REFERENCE TO RELATED APPLICATIONS

This application claims priority to U.S. Provisional Application Ser. No. 61/484,787 entitled “Systems and Methods for Producing an Evaporation Barrier in a Reaction Chamber,” filed on May, 11, 2011, which is incorporated herein by reference in its entirety.

### BACKGROUND

The embodiments described herein relate to systems and methods for producing an evaporation barrier in a reaction chamber. More particularly, some embodiments described herein can be applied to reaction chambers for nucleic acid isolation and amplification.

Some known diagnostic procedures include the isolation and analysis of nucleic acids, such as DNA or RNA. Known methods for isolating nucleic acids within a sample often include several steps, such as: (1) removing the proteins within the sample by adding a protease (e.g., Proteinase K); (2) breaking down the remaining bulk sample to expose the nucleic acids contained therein (also referred to as cell lysing); (3) precipitating the nucleic acid from the sample; and (4) washing and/or otherwise preparing the nucleic acid for further analysis.

In some instances, amplification of the isolated nucleic acid (e.g., replication of the nucleic acid to increase its volume) is desired for further analysis. The polymerase chain reaction (PCR) process is a known technique for amplifying portions of a nucleic acid molecule. During a PCR, an input sample containing the target DNA is mixed with liquid reagents, which can include the DNA polymerase (e.g., the Taq polymerase). The input sample can be, for example, the isolated nucleic acid sample produced by the procedure described above. The liquid reaction mixture is then thermally cycled multiple times within an isolated reaction chamber to complete the reaction. The temperatures and time periods of the thermal cycling are carefully controlled to ensure accurate results. After the DNA sequence is sufficiently amplified, it can be analyzed using various optical techniques.

In some known systems for performing nucleic acid amplification, a portion of the liquid reaction mixture containing the input sample and the reagents can evaporate into the air volume contained in the isolated reaction chamber during the PCR process. Such evaporation can affect the relative concentrations or proportion of the different components in the liquid reaction mixture, and can affect the reaction and/or the optical monitoring of the reaction. For example, evaporation of a portion of the reaction mixture can result in decreased uniformity of amplification. In addition, the evaporated reaction mixture can condense on the walls of the reaction chamber not otherwise covered by the liquid reaction mixture. Such condensation on the walls of the reaction chamber can also affect the optical monitoring or analysis of the reaction, or otherwise affect the operation of the nucleic acid amplification instrument. Thus, it is desirable to reduce the evaporation of the liquid reaction mixture during the PCR process.

Some known methods for reducing evaporation in nucleic acid isolation and amplification systems include applying a layer of mineral oils to the reaction mixture to serve as an evaporation barrier at the surface of the liquid reaction mixture. However, the use of mineral oils as and evaporation barrier can introduce impurities, which can affect the batch- to-batch consistency of the reaction. The properties of mineral oils can also change as the number of thermal cycles increase. In addition, the application of such mineral oils as an evaporation barrier can be costly and/or require additional steps to implement in automated nucleic acid isolation and amplification systems.

Thus, a need exists for improved systems and methods for producing an evaporation barrier in a reaction chamber.

### SUMMARY

The embodiments described herein relate to systems and methods for producing an evaporation barrier in a reaction chamber. In some embodiments, an apparatus for performing a polymerase chain reaction includes a sample isolation module, a storage module, and a reaction module. The sample isolation module defines a chamber configured to include a sample. The storage module includes a transfer mechanism that defines a volume containing multiple particles formulated to be buoyant when disposed in the sample. The storage module can be physically and fluidically coupled to the sample isolation module such that the particles are transferred to the chamber when then the transfer mechanism is actuated. The reaction module includes a transfer mechanism and defines a reaction chamber. The reaction module can be physically and fluidically coupled to the sample isolation module such that the reaction chamber is in fluid communication with the chamber of the sample isolation module. The sample and the multiple particles are transferred from the chamber of the sample isolation module to the reaction chamber when the transfer mechanism of the reaction module is actuated.

### BRIEF DESCRIPTION OF THE DRAWINGS

FIG. 1 is a side perspective view of a cartridge for use in a PCR process, according to an embodiment.

FIG. 2 is a side perspective view of an isolation module of the cartridge shown in FIG. 1, in a first configuration.

FIG. 3 is a side cross-sectional view of the isolation module shown in FIG. 2, in the first configuration.

FIG. 4 is a side cross-sectional view of the isolation module shown in FIG. 2, in a second configuration.

FIG. 5 is a side perspective view of PCR module of the cartridge shown in FIG. 1, in a first configuration.

FIG. 6 is a side cross-sectional view of the PCR module shown in FIG. 5, in the first configuration.

FIG. 7 is a side cross-sectional view of the PCR module shown in FIG. 5, in a second configuration.

FIGS. 8 and 9 are side cross-sectional views of the cartridge shown in FIG. 1, in a first configuration and a second configuration, respectively.

FIG. 10 is an instrument configured to engage the cartridge of FIGS. 1-9 during a PCR process, according to an embodiment.

FIG. 11 is an exploded, partial cross-sectional, schematic illustration of a portion of an instrument, according to an embodiment

FIG. 12 is an illustration of an arrangement of beads forming an evaporation barrier, according to an embodiment.

FIG. 13 is a cross-sectional perspective view of a cartridge for use in a PCR process, according to an embodiment.

FIG. 14 is an enlarged view of a flow cell included in the cartridge of FIG. 13.

FIG. 15 shows a graph comparing the number of thermal cycles at the point of detecting the amplification of nucleic acid in PCR experiments applying various evaporation barriers.

FIG. 16 shows a graph comparing the anneal temperatures in PCR experiments applying various evaporation barriers.

FIG. 17 is a flowchart illustrating a method of establishing an evaporation barrier during a PCR process, according to an embodiment.

FIG. 18 is a flowchart illustrating a method of establishing an evaporation barrier during a PCR process, according to an embodiment.

### DETAILED DESCRIPTION

In some embodiments, an apparatus for performing a polymerase chain reaction includes a sample isolation module, a storage module, and a reaction module. The sample isolation module defines a chamber configured to include a sample. The storage module includes a transfer mechanism that defines a volume containing multiple particles configured to be buoyant when disposed in the sample. The storage module can be physically and fluidically coupled to the sample isolation module such that the multiple particles are transferred to the chamber when then the transfer member is actuated. The reaction module includes a transfer mechanism and defines a reaction chamber. The reaction module can be physically and fluidically coupled to the sample isolation module such that the reaction chamber is in fluid communication with the chamber of the sample isolation module. The sample and the multiple particles are transferred from the chamber of the sample isolation module to the reaction chamber when the transfer mechanism of the reaction module is actuated.

In some embodiments, an apparatus for performing a polymerase chain reaction includes a storage module and a reaction module. The storage module includes a first transfer mechanism and defines a volume. The volume contains multiple particles formulated to be buoyant when disposed in a sample. The reaction module includes a second transfer mechanism and defines a reaction chamber. The reaction module is configured to be coupled to a sample isolation module such that the sample is transferred from sample isolation module to the reaction chamber when the second transfer mechanism is actuated. The storage module is configured to be coupled to the reaction module such that the volume is selectively placed in fluid communication with the reaction chamber. The first transfer mechanism is configured to transfer the multiple particles from the volume to the reaction chamber when the first transfer mechanism is actuated.

In some embodiments, a method includes conveying a sample containing a target nucleic acid into a reaction vial containing a series of particles. An evaporation barrier is formed along a top surface of the sample when the sample is disposed within the reaction vial, the evaporation barrier including the series of particles. The method further includes performing a polymerase chain reaction on the sample within the reaction vial and analyzing the sample, after the polymerase chain reaction, within the reaction vial while the evaporation barrier is present along the top surface of the sample.

In some embodiments, a method includes conveying a sample containing a target nucleic acid into a reaction vial. The method further includes conveying multiple particles from a storage module into the reaction vial. An evaporation barrier is formed along a top surface of the sample within the reaction vial, the evaporation barrier including the multiple particles. The method further includes performing a polymerase chain reaction on the sample within the reaction vial. In some embodiments, the method optionally includes binding a target nucleic acid within the sample to a portion of the multiple particles. In such embodiments, the portion of the particles can be formulated to produce a light emission, and can therefore be used to assist in optical detection of the target nucleic acid.

Apparatus and methods for producing an evaporation barrier described herein can be used in conjunction with any suitable polymerase chain reaction (PCR) system that produces data (e.g., fluorescence output or the like), including the system shown in U.S. Patent Publication No. 2011/0236960, entitled, “Apparatus and Methods for Integrated Sample Preparation, Reaction, and Detection,” filed Feb. 23, 2011, which is incorporated herein by reference in its entirety.

The PCR, in one embodiment, uses an isolated or purified nucleic acid as the template. “Purified nucleic acid” and “isolated nucleic acid” each refer to a sample comprising at least about 70% nucleic acid, at least about 75% nucleic acid, at least about 80% nucleic acid, at least about 85% nucleic acid, at least about 90% nucleic acid, at least about 95% nucleic acid, at least about 96% nucleic acid, at least about 97% nucleic acid, at least about 98% nucleic acid or at least about 99% nucleic acid. The purified or isolated nucleic acid can comprise solely RNA, solely DNA, or a combination thereof. A “purified DNA” or “isolated DNA” refers to a sample comprising at least about 70% DNA, at least about 75% DNA, at least about 80% DNA, at least about 85% DNA, at least about 90% DNA, at least about 95% DNA, at least about 96% DNA, at least about 97% DNA, at least about 98% DNA or at least about 99% DNA. Similarly, “purified RNA” or “isolated RNA” refers to sample comprising at least about 70% RNA, at least about 75% RNA, at least about 80% RNA, at least about 85% RNA, at least about 90% RNA, at least about 95% RNA, at least about 96% RNA, at least about 97% RNA, at least about 98% RNA or at least about 99% RNA.

A “target nucleic acid,” or “target,” as used herein, is a specific nucleic acid sequence whose presence or absence is probed for during a PCR. For example, in one embodiment, the target nucleic acid is a viral nucleic acid, e.g., an Influenza RNA. In one embodiment, at least one or at least two target nucleic acids are probed for during a PCR. If the target is an RNA, prior to the PCR, a reverse transcription reaction is carried out on the RNA.

In one embodiment, substantially the entire isolated or purified nucleic acid sample is used in the subsequent PCR reaction, as described in detail in U.S. Provisional Application No. 61/529,794, entitled “Nucleic Acid Amplification Methods Using Mechanical Hot Start,” filed Aug. 31, 2011, the disclosure of which is incorporated by reference in its entirety.

In one embodiment, the “purified nucleic acid” or “isolated nucleic acid” is purified or isolated from a clinical sample, e.g., a nasopharyngeal sample. In a further embodiment, the purified nucleic acid comprises is a viral nucleic acid, e.g., as the target nucleic acid.

As indicated above, the nucleic acid sample used in the PCR is a purified DNA sample, a purified RNA sample, or a combination thereof. The nucleic acid, in one embodiment, is originally present in a heterogeneous biological sample, for example a reconstituted nasopharyngeal sample. The target nucleic acid, present in the purified nucleic acid sample, is either at least one DNA or RNA sequence. In one embodiment, the at least one target includes at least two target nucleic acid sequences. In one embodiment, the target nucleic acid comprises at least one viral nucleic acid, i.e., viral DNA or viral RNA, or a combination thereof. In one embodiment, the PCR is used to simultaneously detect the presence or absence of an influenza A RNA molecule, influenza B RNA molecule and respiratory syncytial virus RNA molecule. Other target nucleic acids are provided in U.S. application Ser. No. 13/464,240, entitled “Apparatus and Methods for Integrated Sample Preparation, Reaction and Detection,” filed on May 4, 2012, which is incorporated herein in its entirety.

During and/or after the PCR, the presence or absence of a target nucleic acid, in one embodiment, is detected by a fluorescence measurement. For example, in one embodiment, PCR is monitored using DNA probes known as “molecular beacons” (Tyagi et al., Nat. Biotech., 16: 49-53 (1998)). Molecular beacons have a hairpin structure wherein the quencher dye and reporter dye are in intimate contact with each other at the end of the stem of the hairpin. Upon hybridization with a complementary sequence (i.e., the target nucleic acid), the loop of the hairpin structure becomes double stranded and forces the quencher and reporter dye apart, thus generating a fluorescent signal. Accordingly, an amount of target nucleic acid is directly proportional to the amount of fluorescence generated.

In another embodiment, the PCR is monitored in real time with the use of a fluorescent probe, for example, a single stranded DNA molecule comprising a minor groove binder (MGB) and a fluorophore at the 5′ end, and a non-fluorescent quencher at its 3′-end (for example, see U.S. Pat. Nos. 5,801,115 and 6,727,356, both incorporated herein by reference in their entireties). The MGB molecule, conjugated to the oligonucleotide probe, in one embodiment, serves multiple functions. When the oligonucleotide probe is not hybridized to a target, the MGB molecule acts as an additional quencher to the attached fluorophore enhancing the signal-to-background ratio for test results. When the probe is hybridized to a target, the MGB molecule fits into the minor groove of the double helix structure enhancing the bond between the oligonucleotide probe and the target nucleic acid. In the bound configuration, fluorescence is no longer quenched by either the MGB or the quencher molecule. In this regard, the amount of target nucleic acid is directly proportional to the amount of fluorescence generated.

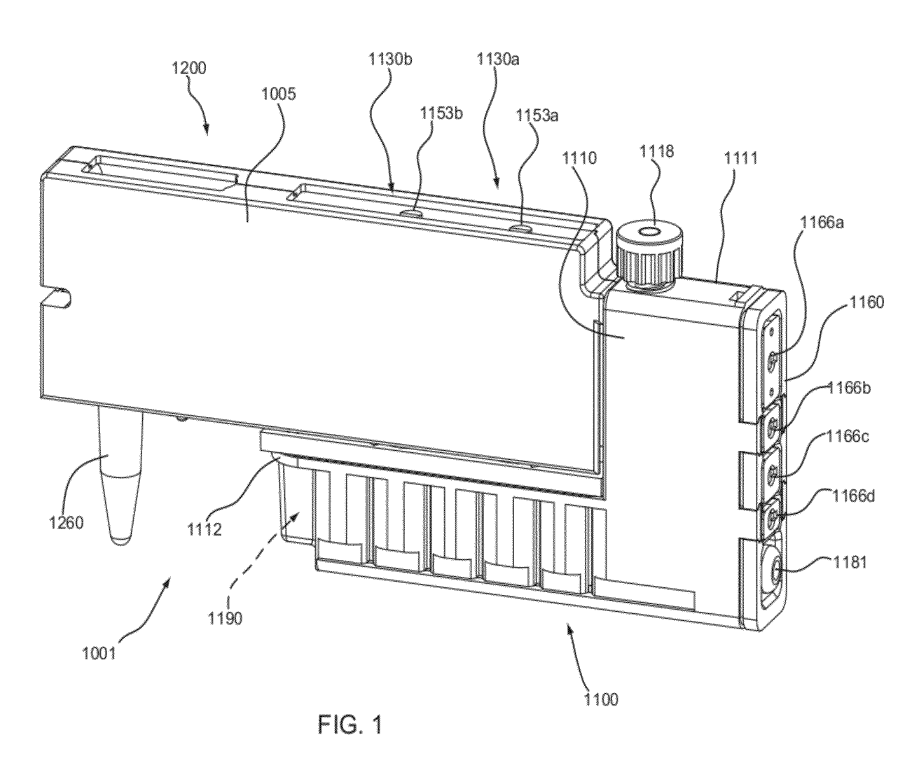

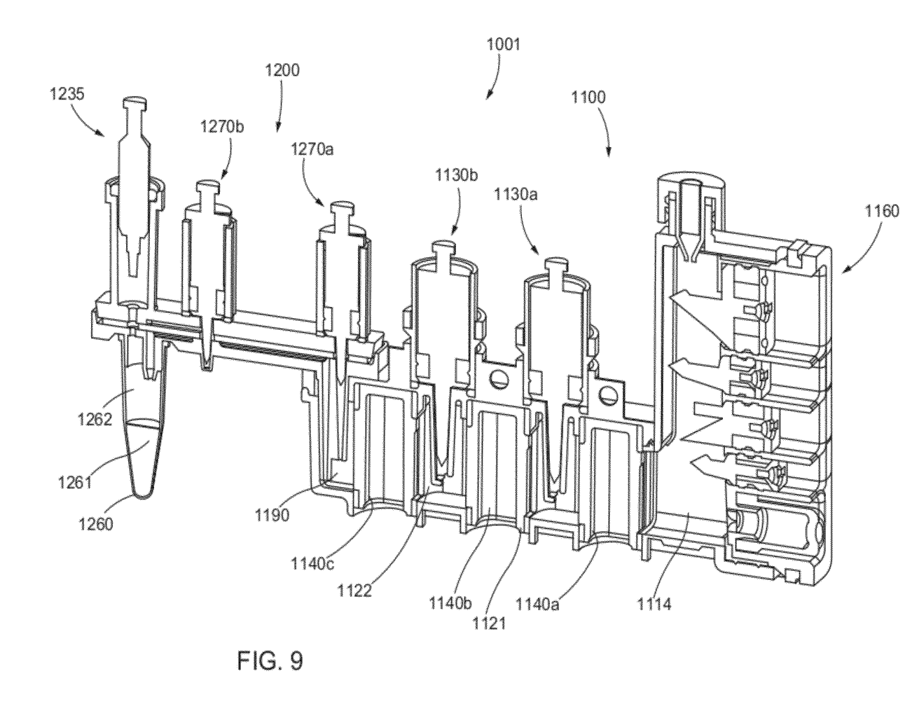

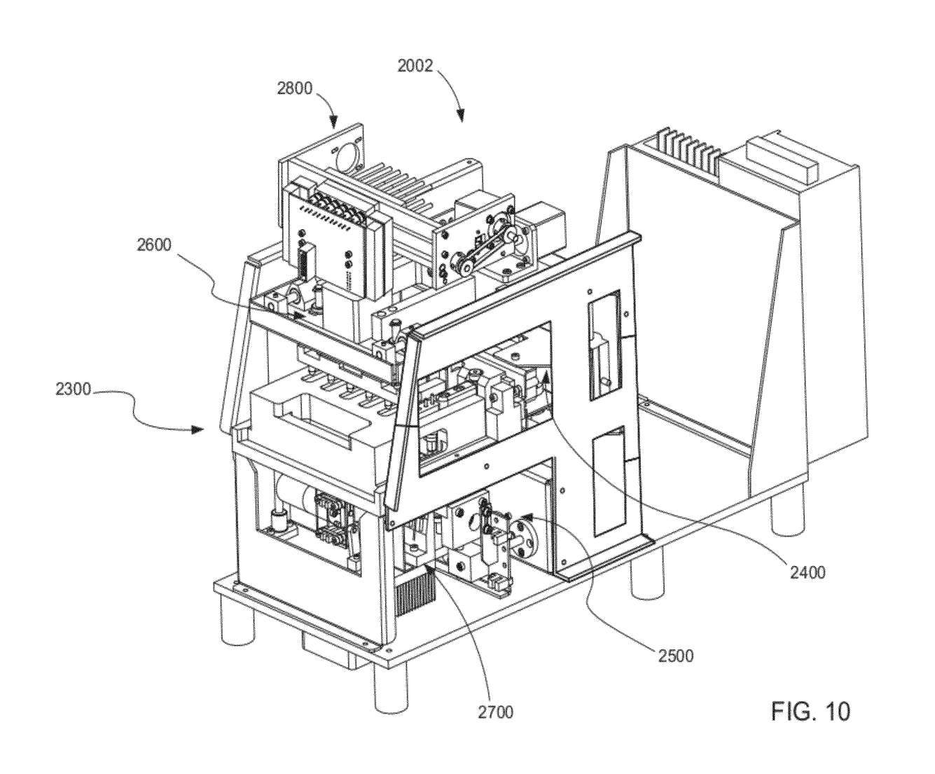

In some embodiments, the apparatus and methods for producing an evaporation barrier in PCR procedures shown and described herein can be used on a system of the type shown in FIGS. 1-10. As described in further detail herein, FIG. 1-9 show a cartridge 1001 within which a nucleic acid of a sample can be isolated or purified, and upon which a PCR and/or melt curve assay can be performed. FIG. 10 shows an instrument configured to manipulate one or more cartridges of the type shown in FIGS. 1-9 to perform a PCR and/or melt curve assay to produce data of the types described herein.

As shown in FIG. 1, the cartridge 1001 includes a sample preparation (or isolation) module 1100 and an amplification (or PCR) module 1200 that are coupled together to form an integrated cartridge 1001. A cover 1005 is disposed about a portion of the isolation module 1100 and the PCR module 1200. One or more cartridges 1001 can be disposed within any suitable instrument of the types disclosed in U.S. Patent Publication No. 2011/0236960, and below with reference to FIG. 10 (e.g., an instrument 2002) that is configured to manipulate, actuate and/or interact with the cartridge 1001 to perform a nucleic acid isolation, transcription and/or amplification on a test sample contained within the cartridge.

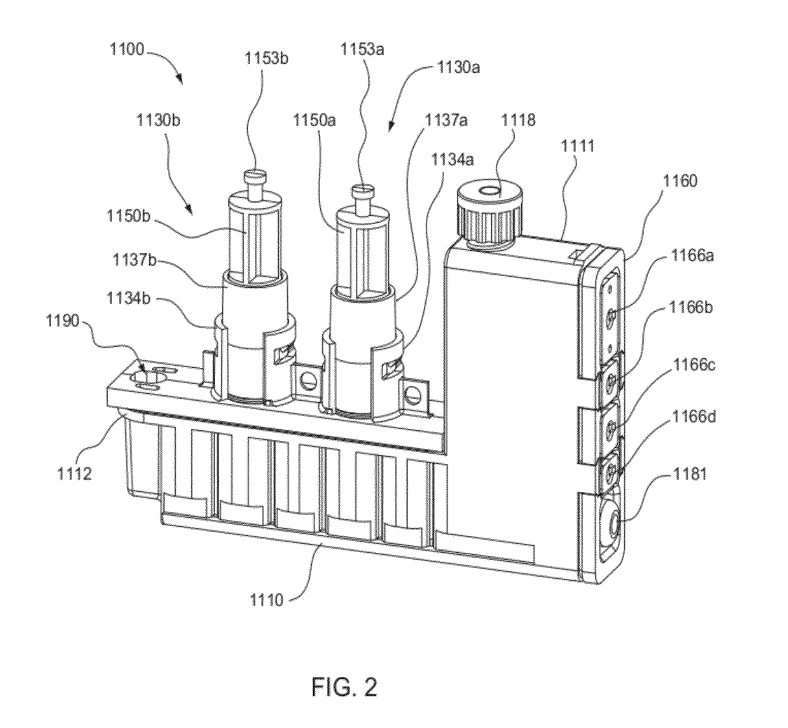

As shown in FIGS. 2-4, the isolation module 1100 includes a first (or isolation) housing 1110 and a second (or reagent) housing 1160 that is coupled to the first housing 1110. The second housing 1160 defines a series of holding chambers 1163a, 1163b, 1163c and 1163d that contain the reagents and/or other substances used in the isolation process. The holding chambers can contain, for example, a protease (e.g., Proteinase K), a lysis solution to solubilize the bulk material, a binding solution to magnetically charge the nucleic acid sample resident within a lysing chamber 1114, and a solution of magnetic beads that bind to the magnetically charged nucleic acid to assist in the conveyance of the nucleic acid within the isolation module 1100 and/or the first housing 1110.

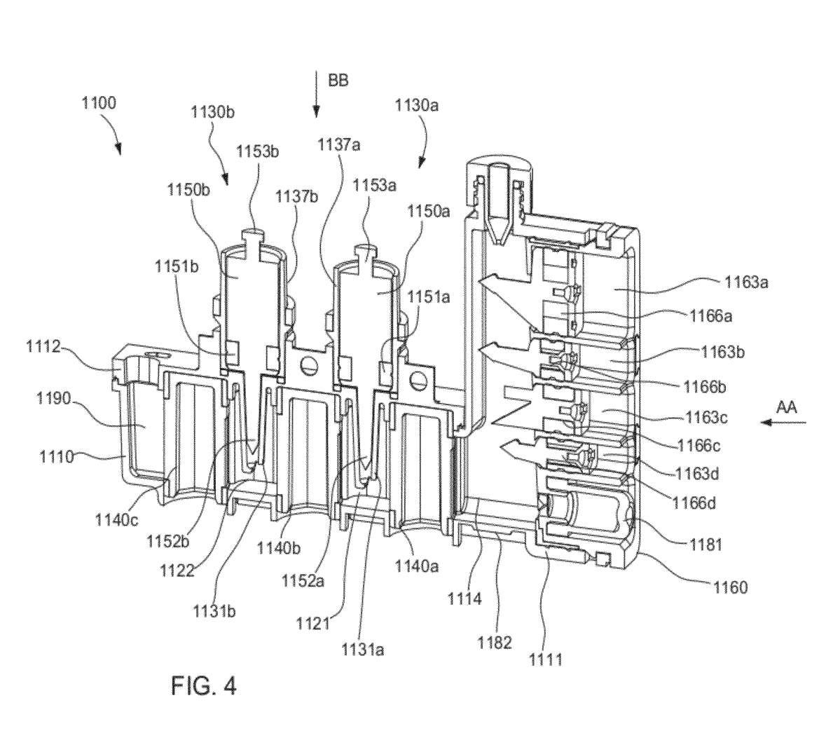

Each of the holding chambers 1163a, 1163b, 1163c and 1163d includes an actuator movably disposed therein. More particularly, an actuator 1166a is disposed within the holding chamber 1163a, an actuator 1166b is disposed within the holding chamber 1163b, an actuator 1166c is disposed within the holding chamber 1163c, and an actuator 1166d is disposed within the holding chamber 1163d. Each of the actuators 1166a, 1166b, 1166c and 1166d can function as a transfer mechanism to convey substances from the chamber (e.g., chamber 1163a) into another portion of the isolation module 1100 when moved in the direction of the arrow AA in FIG. 4.

The second housing 1160 includes a mixing pump 1181, which can be actuated (e.g., by the actuator assembly 2400 of the instrument 2002) to agitate, mix and/or produce a turbulent motion within the sample, reagents and/or other substances contained with a portion of the isolation module 1100. The arrangement of the holding chambers 1163a, 1163b, 1163c and 1163d, the actuators 1166a, 1166b, 1166c and 1166d, and the mixing pump 1181 allow the substances contained within the second housing 1160 to be conveyed into the first housing 1110 when the actuators 1166a, 1166b, 1166c and 1166d are actuated.

The first housing 1110 includes a first portion 1112 and a second portion 1111. The first housing 1110 defines the lysing chamber 1114, two wash chambers 1121 and 1122, three transfer assembly 1140a, 1140b, and 1140c, and an elution chamber 1190. The isolation module 1100 includes a cap 1118 that is removably coupled to the housing 1110. In use, a sample containing a target nucleic acid, such as, for example, urine, blood and/or other materials containing tissue samples can be conveyed into the lysing chamber 1114 upon removal of the cap 1118. After the sample is disposed into the lysing chamber 1114, reagents and/or substances to facilitate cell lysis can be added to the lysing chamber 1114, as described above. Moreover, the sample can be agitated and/or mixed via the pump 1181 to facilitate the lysing process. While not shown in FIG. 1, in some embodiments, the first housing 1100 can include a portion that receives an ultrasonic transducer configured to further facilitate cell lysis (for example, of the types described in U.S. Non-Provisional patent application Ser. No. 13/464,240, entitled, “Apparatus and Methods for Integrated Sample Preparation, Reaction, and Detection,” filed May 4, 2012, which is incorporated herein by reference in its entirety).

The transfer assemblies 1140a, 1140b, and 1140c are configured to transfer substances (e.g., portions of the sample including the magnetically charged particles and the isolated nucleic acid attached thereto) between the lysing chamber 1114, the wash chamber 1121, the wash chamber 1122, and the elution chamber 1190. The wash chamber 1121 and 1122 receive a wash buffer module 1130a and 1130b, respectively configured to convey a wash buffer solution (a mineral oil and/or any other substance to be added to the sample). The wash chambers 1121 and 1122 and the wash buffer modules 1130a and 1130b are configured to promote washing and or mixing of the portion of the sample contained therein.

The wash buffer module 1130a includes an actuator 1150a that is movably disposed within a housing 1137a. The housing 1137a is coupled to the upper portion 1112 of the first housing 1110 such that the wash buffer module 1130a is substantially aligned with the wash chamber 1121. In particular, the housing 1137a includes a pair of protrusions 1133a that are configured to be disposed within a corresponding opening defined by a coupling portion 1134a of the upper portion 1112 of the first housing 1110.

The actuator 1150a includes a plunger portion 1151a, a piercing portion 1152a and an engagement portion 1153a. The engagement portion 1153a is configured to engage with, be removably coupled to and/or be received within a portion of an actuator assembly to facilitate movement of the actuator 1150a within the housing 1137a, as described herein. The actuator 1150a can be manipulated and/or actuated by any suitable instrument, such as the actuator assembly 2600 described below with respect to FIG. 10.

The plunger portion 1151a of the actuator 1150a is disposed within the housing 1137a. A puncturable member 1135a is disposed about the end portion of the housing 1137a such that end face of the plunger portion 1151a, the housing 1137a and the puncturable member 1135a collectively define a volume within which a substance is disposed. The plunger portion 1151a and the internal surface of the housing 1137a are configured to form a substantially fluid-tight and/or hermetic seal. In some embodiments, the plunger portion 1151 a can include a sealing member, an o-ring or the like.

The piercing portion 1152a of the actuator 1150a is configured to puncture, break, sever and/or rupture a portion of the puncturable member 1135a when the actuator 1150a is moved within the housing 1137a in the direction indicated by the arrow BB in FIG. 4. In this manner, movement of the actuator 1150 places the chamber in fluid communication with the wash chamber 1121. Similarly stated, wash buffer module 1130a can be selectively placed in fluid communication with the wash chamber 1121 when the actuator 1150a is actuated. After the substance within the wash buffer module 1130a is conveyed into the wash chamber 1121, the actuator 1150a can be reciprocated within the housing 1137a to produce a pressure that is conveyed into the wash chamber 1121 to promote washing, mixing and/or other interaction between and with the sample disposed therein. The top portion 1112 of the first housing 1110 includes a nozzle 1131a configured to direct the pressure energy and/or flow produced by the actuator 1150a towards a particular region within the wash chamber 1121.

The wash buffer module 1130b includes an actuator 1150b that is movably disposed within a housing 1137b. The housing 1137b is coupled to the upper portion 1112 of the first housing 1110 such that the wash buffer module 1130b is substantially aligned with the wash chamber 1122. In particular, the housing 1137b includes a pair of protrusions 1133b that are configured to be disposed within a corresponding opening defined by a coupling portion 1134b of the upper portion 1112 of the first housing 1110.

The actuator 1150b includes a plunger portion 1151b, a piercing portion 1152b and an engagement portion 1153b. The engagement portion 1153b is configured to engage with, be removably coupled to and/or be received within a portion of an actuator assembly to facilitate movement of the actuator 1150b within the housing 1137b, as described herein. The plunger portion 1151b of the actuator 1150b is disposed within the housing 1137b. A puncturable member 1135b is disposed about the end portion of the housing 1137b such that end face of the plunger portion 1151b, the housing 1137b and the puncturable member 1135b collectively define a volume within which a substance is disposed. The plunger portion 115 1b and the internal surface of the housing 1137b are configured to form a substantially fluid-tight and/or hermetic seal.

The piercing portion 1152b of the actuator 1150b is configured to puncture, break, sever and/or rupture a portion of the puncturable member 1135b when the actuator 1150b is moved within the housing 1137b in the direction indicated by the arrow BB in FIG. 4. In this manner, movement of the actuator 1150b places the chamber in fluid communication with the wash chamber 1122. Similarly stated, wash buffer module 1130b can be selectively placed in fluid communication with the wash chamber 1122 when the actuator 1150b is actuated. After the substance within the wash buffer module 1130b is conveyed into the wash chamber 1122, the actuator 1150b can be reciprocated within the housing 1137b to produce a pressure that is conveyed into the wash chamber 1122 to promote washing, mixing and/or other interaction between and with the sample disposed therein. The top portion 1112 of the first housing 1110 includes a nozzle 1131b configured to direct the pressure energy and/or flow produced by the actuator 1150b towards a particular region within the wash chamber 1122.

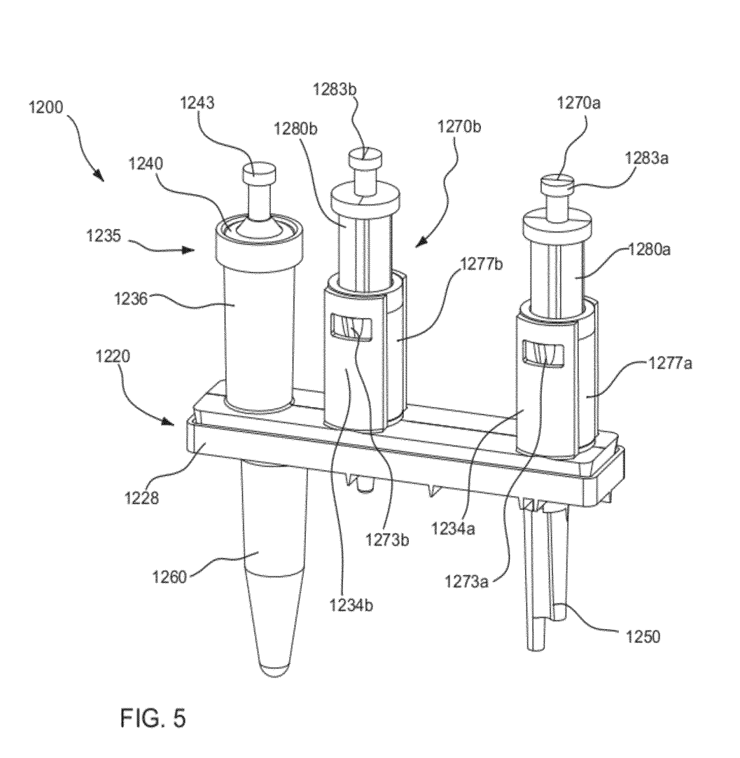

As shown in FIGS. 5-7, the amplification (or PCR) module 1200 includes a substrate 1220 that is constructed from a first (or upper) layer 1227 and a second (or bottom) layer 1228. The PCR module 1200 includes a PCR vial 1260 coupled to the second layer 1228, a transfer mechanism 1235, a first reagent module 1270a and a second reagent module 1270b. The PCR vial 1260 is coupled to the first end portion 1211 of the housing 1210 and defines a volume within which a sample can be disposed to facilitate a reaction associated with the sample. The PCR vial 1260 can be any suitable container for containing a sample in a manner that permits a reaction associated with the sample to occur. The PCR vial 1260 can also be any suitable container for containing the sample in a manner that permits the monitoring of such a reaction (e.g., the detection of an analyte within the sample that results from or is associated with the reaction). In some embodiments, at least a portion of the PCR vial 1260 can be substantially transparent to allow optical monitoring of a reaction occurring therein be an optical system (e.g., the optics assembly 2800 of the instrument 2002 described herein).

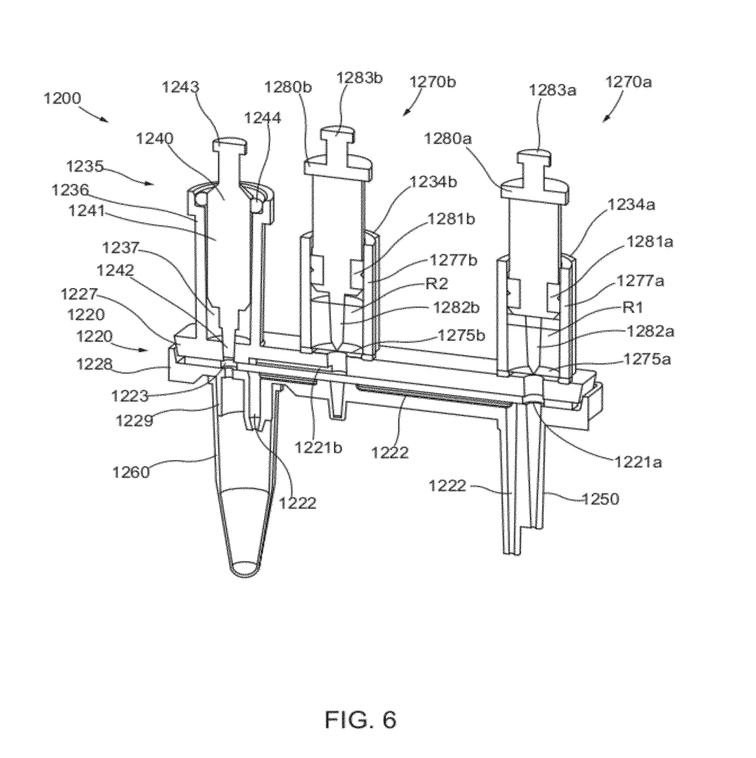

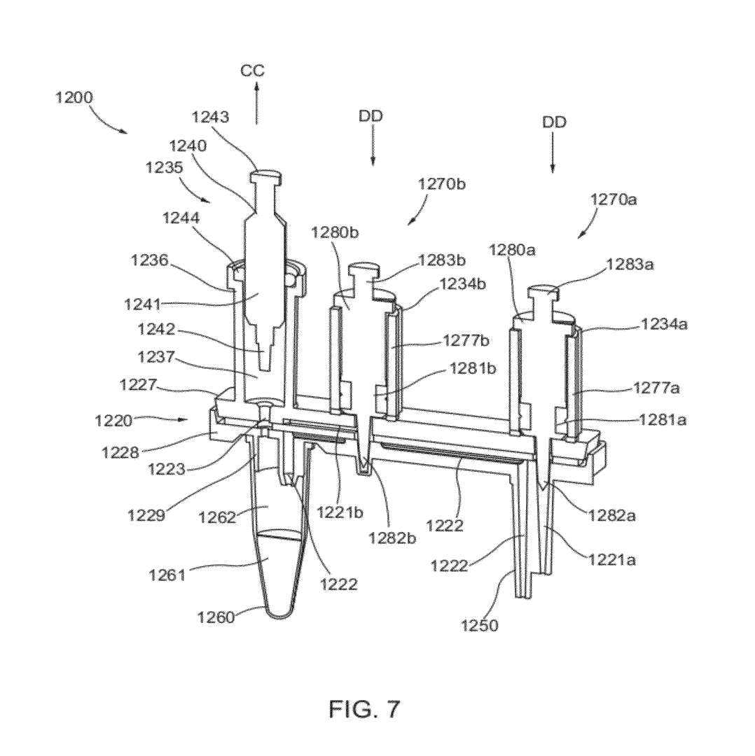

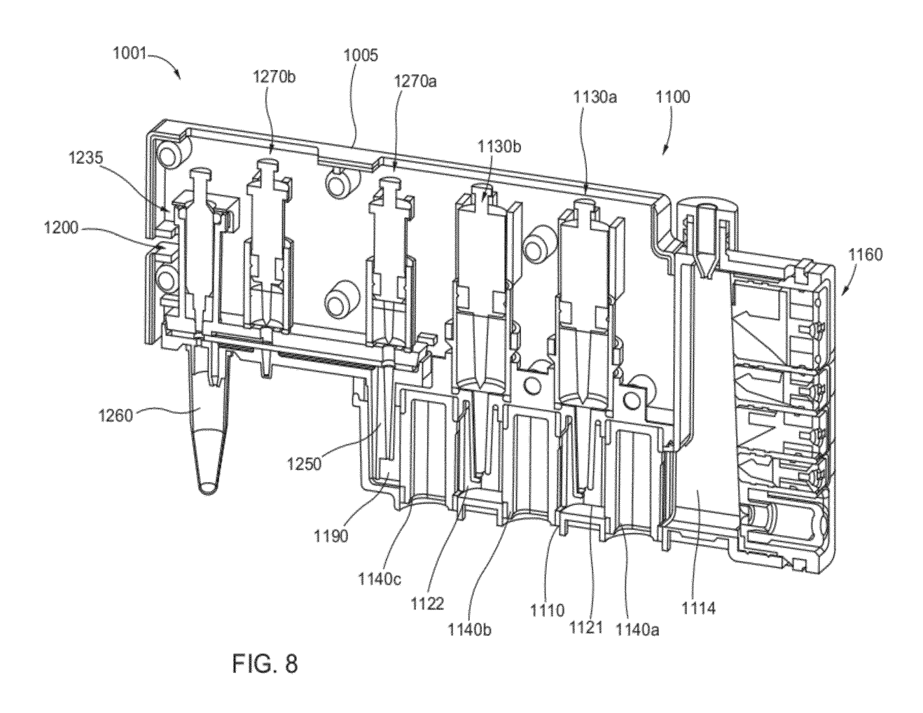

As shown in FIGS. 8 and 9, the amplification module 1200 is coupled to the first housing 1110 of the isolation module 1100 such that at least a portion of a transfer tube 1250 is disposed within the elution chamber 1190 of the isolation module 1100. In this manner, as described herein, the isolated nucleic acid, any substances and/or any PCR reagents disposed within the elution chamber 1190 can be conveyed from the elution chamber 1190 to the PCR vial 1260 via the transfer tube 1250. More particularly, the substrate 1220 defines a flow passageway 1222 that places the PCR vial 1260 in fluid communication with the elution chamber 1190 when the PCR module 1200 is coupled to the isolation module 1100. As shown in FIGS. 6 and 7, portions of the flow passageway 1222 are defined in the transfer tube 1250 and a transfer port 1229 of the second layer 1228 of the substrate 1220.

The substrate 1220 also defines a flow passageway 1223, a flow passageway 1221a and a flow passageway 1221b. As described in more detail herein, the flow passageway 1223 is configured to place a volume 1237 defined within the transfer mechanism 1235 in fluid communication with the PCR vial 1260 via the transfer port 1229. The flow passageway 1221a is configured to place a volume defined by the reagent module 1270a in fluid communication with the elution chamber 1190 via the transfer tube 1250. The flow passageway 1221b is configured to place a volume defined by the reagent module 1270b in fluid communication with the PCR vial 1260 via the transfer port 1229 and/or a portion of the passageway 1222. Any of the flow passageway 1223, the flow passageway 1221a and/or the flow passageway 1221b can be defined by the first layer 1227, the second layer 1228, or in portions of both the first layer 1227 and the second layer 1228. In addition, the flow passageway 1223, the flow passageway 1221a and/or the flow passageway 1221b can be any suitable shape or configuration. In some embodiments, the flow passageways can have a constant diameter. In other embodiments, any of the flow passageways can include a step or taper such that a given diameter is reduced, as further described herein.

The PCR module 1200 includes two reagent modules 1270a and 1270b that are each coupled to the upper layer 1227 of the substrate 1220. As described herein, each reagent module 1270a and 1270b contains a substance, R1 and R2, respectively. The reagent module 1270a is configured to convey the substance R1 into the elution chamber 1190 via the flow passageway 1221a, as described herein. The reagent module 1270b is configured to convey the substance R2 into the PCR vial 1260 via the flow passageway 1221b, as described herein. In this manner, each reagent module 1270a and 1270b functions as a reagent storage device and a transfer mechanism.

The substances R1 and R2 can be, for example, a reagent, an elution buffer solution, a wash buffer solution, a mineral oil and/or any other substance to be added to the sample, as described herein. In some embodiments, the substance R1 can include an elution buffer and mineral oil. In some embodiments, the substance R2 can include reaction reagents that facilitate a PCR process within the PCR vial 1260. In some embodiments, a PCR master mix can be disposed within the PCR vial 1260 in a lyophilized state such that the addition of the substance R2 and/or a mixture of the substance R1 and the target sample reconstitutes the lyophilized master mix to facilitate the PCR process. In some embodiments, the substance R1 and/or R2 can include constituents configured to form an evaporation barrier such as beads or microspheres, as described in further detail herein.

In some embodiments, a master mix comprises lyophilized reagents to perform a multiplex PCR on three targets and an internal control. In a further embodiment, the target nucleic acids are a nucleic acid specific for influenza A, a nucleic acid specific for influenza B and a nucleic acid specific for RSV. In even a further embodiment, the multiplex reaction is monitored in real time, for example, by providing a hybridizing oligonucleotide probe, specific for each target sequence, each probe comprising a fluorophore and MGB at the 5′-end and a non-fluorescent quencher at the 3′ end.

In another embodiment, the lyophilized master mix comprises reagents for both a PCR and a reverse transcriptase reaction. For example, in one embodiment, the lyophilized master mix includes both the reverse transcriptase and Taq polymerase enzymes, dNTPs, RNase inhibitor, KCl, BSA and primers to carry out first strand cDNA synthesis and PCR.

The master mix comprises different primers and probes, depending on the target to be amplified. Each target will have associated with it a specific primer and probe set, and the primer and probe set can be lyophilized with the other PCR reagents mentioned above, to form a lyophilized master mix. Concentrations of components will also vary depending on the particular target being amplified, and if multiple targets are amplified.

In some embodiments, the master mix includes constituents configured to form an evaporation barrier within the PCR vial 1260 during a PCR process. For example, in some embodiments, the master mix includes beads or microspheres, as further described herein.

The reagent module 1270a includes an actuator 1280a that is movably disposed within a housing 1277a. The housing 1277a is coupled to the upper layer 1227 of the substrate 1220 such that the reagent module 1270a is substantially aligned with the passageway 1221a, the transfer tube 1250 and/or the elution chamber 1190. As shown in FIG. 5, the housing 1277a includes a pair of protrusions 1273a that are configured to be disposed within a corresponding opening defined by a coupling portion 1234a of the upper layer 1227 of the substrate 1220.

The actuator 1280a includes a plunger portion 1281a, a piercing portion 1282a and an engagement portion 1283a. The engagement portion 1283a is configured to engage with, be removably coupled to and/or be received within a portion of an actuator assembly (e.g., included in the instrument 2002) to facilitate movement of the actuator 1280a within the housing 1277a, as described herein.

The plunger portion 1281a of the actuator 1280a is disposed within the housing 1277a. A puncturable member 1275a is disposed about the end portion of the housing 1277a such that end face of the plunger portion 1281a, the housing 1277a and the puncturable member 1275a collectively define a volume within which the substance R1 is disposed. The plunger portion 1281a and the internal surface of the housing 1277a are configured to form a substantially fluid-tight and/or hermetic seal. In some embodiments, the plunger portion 1281a can include a sealing member, an o-ring or the like.

The piercing portion 1282a of the actuator 1280a is configured to puncture, break, sever and/or rupture a portion of the puncturable member 1275a when the actuator 1280a is moved within the housing 1277a in the direction indicated by the arrow CC in FIG. 7. In this manner, movement of the actuator 1280a places the volume therein in fluid communication with the passageway 1221a, and therefore the elution chamber 1190. Similarly stated, reagent module 1270a can be selectively placed in fluid communication with the elution chamber 1190 when the actuator 1280a is actuated.

The reagent module 1270b includes an actuator 1280b that is movably disposed within a housing 1277b. The housing 1277b is coupled to the upper layer 1227 of the substrate 1220 such that the reagent module 1270b is substantially aligned with the passageway 1221b. As shown in FIG. 5, the housing 1277b includes a pair of protrusions 1273b that are configured to be disposed within a corresponding opening defined by a coupling portion 1234b of the upper layer 1227 of the substrate 1220. The actuator 1280b includes a plunger portion 1281b, a piercing portion 1282b, and an engagement portion 1283b that are substantially similar in form and function as the plunger portion 1281a, the piercing portion 1282a, and the engagement portion 1283a. Furthermore, the housing 1277b includes a puncturable member 1275b, configured to be punctured when the actuator 1280b is moved in the direction of the arrow CC in FIG. 7. In this manner, movement of the actuator 1280b places the volume therein in fluid communication with the passageway 1221b, and therefore the PCR chamber 1260.

The PCR module 1200 includes a transfer mechanism 1235 configured to transfer substances from and/or between the elution chamber 1190 of the isolation module 1100 and the PCR vial 1260 of the PCR module 1200. The transfer mechanism 1235 includes an actuator 1240 disposed within a housing 1236. The housing 1236 is coupled to and/or is a portion of the upper layer 1227 of the substrate 1220. The transfer mechanism 1235 is configured to define a volume 1237 within which a substance can be contained, and selectively place the volume 1237 in fluid communication with the PCR vial 1260. In some embodiments, the volume 1237 includes constituents configured to form an evaporation barrier, such as beads or microspheres to be transferred to the PCR vial 1260, as further described herein. Although not shown as including a puncturable member, in some embodiments a portion of the volume 1237 can be surrounded by and/or fluidically isolated by a puncturable member.

The actuator 1240 includes a plunger portion 1241, a valve portion 1242 and an engagement portion 1243. The engagement portion 1243 is configured to engage with, be removably coupled to and/or be received within a portion of an actuator assembly (e.g., included in the instrument 2002 described herein with reference to FIG. 10) to facilitate movement of the actuator 1240 within the housing 1236, as described herein.

The plunger portion 1241 of the actuator 1240 is disposed within the housing 1236. The plunger portion 1241 and the internal surface of the housing 1236 are configured to form a substantially fluid-tight and/or hermetic seal. In some embodiments, the plunger portion 1241 can include a sealing member, an o-ring or the like. Additionally, a seal 1244 is disposed at the top portion of the housing 1236.

The actuator 1240 is configured to be moved within the housing 1236 between a first position (FIG. 6) and a second position (FIG. 7). When the actuator 1240 is in the first position, the valve portion 1242 of the actuator 1240 is disposed at least partially within the flow passageway 1223 such that volume 1237 is substantially fluidically isolated from the flow passageway 1223 and/or the PCR vial 1260. Similarly stated, when the actuator 1240 is in the first position, a portion of the valve portion 1242 is in contact with the upper layer 1227 to produce a substantially fluid-tight and/or hermetic seal. When the actuator 1250 is moved within the housing 1236 in the direction indicated by the arrow DD in FIG. 7, the valve portion 1242 is spaced apart from the upper layer 1227 and/or is removed from the flow passageway 1223, thereby placing the volume 1237 in fluid communication with the passageway 1223, and therefore the PCR chamber 1260. In this manner, when the actuator 1240 is moved, the substance within the volume 1237 can be conveyed into the PCR volume 1262 defined by the PCR vial 1260. For example, in some embodiments, beads or microspheres included in the volume 1237 of the transfer assembly 1235 can be transferred to the PCR vial 1260 when the transfer member 1235 is moved to the second position. In this manner, the beads or microspheres can form an evaporation barrier during a PCR process, as described herein.

Moreover, when the actuator 1240 is moved within the housing 1236, as shown by the arrow DD in FIG. 7, a vacuum is produced within the volume of the PCR vial 1260. This pressure differential between the volume and the elution chamber 1190 results in at least a portion of the contents of the elution chamber 1190 being transferred into the PCR volume 1262 via the transfer tube 1250 and the passageway 1222. In this manner substances and/or samples can be added, mixed and/or conveyed between the elution chamber 1190 and the PCR vial 1260 by actuating the transfer mechanism 1235.

In use, after the one or more target nucleic acids, or population of nucleic acids is isolated and processed within the isolation module 1100, as described above, it is transferred into the elution chamber 1190 via the transfer assembly 1140c. The reagent module 1270a can then be actuated to convey the substance R1 into the elution chamber 1190. For example, in some embodiments, the reagent module 1270a can be actuated to convey a solution containing an elution buffer and mineral oil into the elution chamber 1190. The magnetic beads are then removed (or “washed”) from the nucleic acid by the elution buffer, and removed from the elution chamber 1190 (e.g., by the transfer assembly 1140c). Thus, the elution chamber 1190 contains the isolated and/or purified nucleic acid. In some embodiments, the reagent module 1270a includes constituents configured to form an evaporation barrier, such as beads or microspheres that are conveyed into the elution chamber 1190.

The reagent module 1270b can be actuated to convey the substance R2 into the PCR vial 1260. In some embodiments, the PCR vial 1260 can contain additional reagents and/or substances, such as, for example, a PCR master mix, in a lyophilized state. Accordingly, when the substance R2 is conveyed into the PCR vial 1260, the lyophilized contents can be reconstituted in preparation for the reaction.

The target sample 1261 can be conveyed (either before or after the actuation of the reagent module 1270b described above) from the elution chamber 1190 into the PCR vial 1260 via the transfer tube 1250 and the passageway 1222. In particular, the actuator 1240 of the transfer mechanism 1235 can be actuated to produce a pressure differential within the PCR module 1200 to convey the PCR sample from the elution chamber 1190 into the PCR vial 1260 via the passageway 1222, as described above. In this manner, the PCR sample (the isolated nucleic acid and the PCR reagents) can be partially prepared in the elution chamber 1190. Moreover, when the transfer mechanism 1235 is actuated, the volume 1237 defined therein is placed in fluid communication with the PCR vial 1260 via the passageway 1223, as described above. Thus, in some embodiments, an additional substance (e.g., beads configured to form an evaporation barrier) can be added to the PCR vial 1260 via the same operation as the sample transfer operation.

After the PCR sample 1261 is in the PCR vial 1260, at least a portion of the PCR sample 1261 can be thermally cycled (e.g., via the heating assembly 2700 of the instrument 2002) to perform the desired amplification. Upon completion of and/or during the thermal cycling, the PCR sample can be optically analyzed (e.g., via the optics assembly 2800 of the instrument 2002) to analyze the sample. Alternatively, as described throughout, the PCR sample can be optically analyzed during the PCR, for example, with DNA hybridization probes, each conjugated to an MGB and fluorophore. A description of the instrument 3002, and other suitable instruments for manipulating the cartridge, is provided below.

FIG. 10 is a perspective view of the instrument 2002 configured to manipulate, actuate and/or interact with a series of cartridges (e.g., the cartridge 1001 described above with respect to FIGS. 1-9) to perform a nucleic acid isolation and amplification process on test samples within the cartridges. The instrument 2002 includes a chassis and/or frame 2300, a first actuator assembly 2400, a sample transfer assembly 2500, a second actuator assembly 2600, a heater assembly 2700 and an optics assembly 2800. The frame 2300 is configured to house, contain and/or provide mounting for each of the components and/or assemblies of the instrument 2002 as described herein. The first actuator assembly 2400 is configured to actuate an actuator or transfer mechanism (e.g., the actuators 1166a, 1166b, 1166c, and 1166d) of the isolation module (e.g., isolation module 1100) of the cartridge 1001 to convey one or more reagents and/or substances into the lysing chamber 1114 within the isolation module 1100.

The transfer actuator assembly 2500 is configured to actuate a transfer assembly (e.g. the transfer assemblies 1140a, 1140b, and 1140c) to transfer a portion of the sample between various chambers and/or volumes within an isolation module 1100. The second actuator assembly 2600 is configured to actuate a mixing mechanism (e.g., actuate the engagement portion 1153a of the wash buffer module 1130a and the engagement portion 1153b of the wash buffer module 1130b) of the isolation module 1100 and/or the PCR module (e.g., PCR module 1200) to convey into and/or mix one or more reagents and/or substances within a chamber within the isolation module 1100 and/or the PCR module 1200.

The heater assembly 2700 is configured to heat one or more portions of the cartridge 1001 (e.g., the PCR vial 1260 and/or a region of the housing 1110 adjacent the lysing chamber 1114) to promote and/or facilitate a process within the cartridge 1001 (e.g., to promote, facilitate and/or produce a “hot start” process, a heated lysing process, a PCR process and/or melt curve analysis). The optics assembly 2800 is configured to monitor a reaction occurring with the cartridge 1001. More specifically, the optics assembly 2800 is configured to detect one or more different analytes and/or targets within a test sample in the cartridge 1001 before, during and/or after any of the procedures described herein.

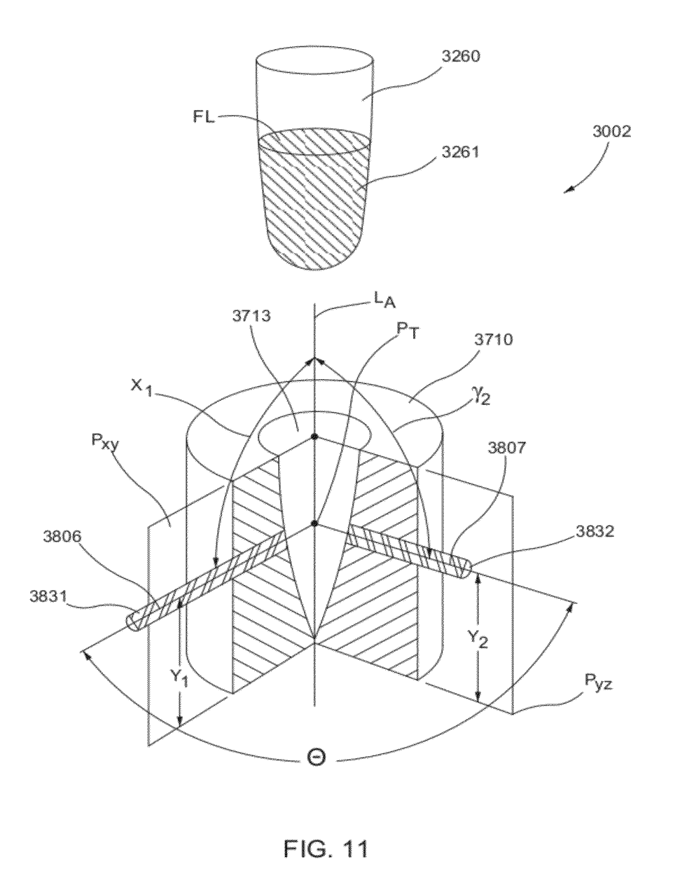

For example, FIG. 11 shows a partial cross-sectional, schematic illustration of a portion of an instrument 3002 according to an embodiment. The instrument 3002, which can be similar to the instrument 2002, includes a block 3710, a first optical member 3831, a second optical member 3832 and an optics assembly (not shown in FIG. 11). The block 3710 defines a reaction volume 3713 configured to receive at least a portion 3261 of a reaction container 3260 that contains a sample 3261. The reaction container 3260 can be any suitable container for containing the sample 3261 in a manner that permits a reaction associated with the sample 3261 to occur, and that permits the monitoring of such a reaction, as described herein. In some embodiments, for example, the reaction container 3260 can be a PCR vial (such as the PCR vial 1260 shown and described above), a test tube or the like. Moreover, in some embodiments, at least the portion 3261 of the reaction container 3260 can be substantially transparent to allow optical monitoring of a reaction occurring therein.

The block 3710 can be any suitable structure for and/or can be coupled to any suitable mechanism for facilitating, producing, supporting and/or promoting a reaction associated with the sample 3261 in the reaction container 3260. For example, in some embodiments, the block 3710 can be coupled to and/or can include a mechanism, such as the heating assembly 2700 of the instrument 2002, for cyclically heating the sample 3261 in the reaction container 3260. In this manner, the block 3710 can produce a thermally-induced reaction of the sample 3261, such as, for example, a PCR process. In other embodiments, the block 3710 can be coupled to and/or can include a mechanism for introducing one or more substances into the reaction container 3260 to produce a chemical reaction associated with the sample 3261.

The reaction volume 3713 can have any suitable size and/or shape for containing the portion 3261 of the reaction chamber 3260. As shown in FIG. 11, the reaction volume 3713 defines a longitudinal axis L_{A} and substantially surrounds the portion 3261 of the reaction chamber 3260 when the portion 3261 is disposed within the reaction volume 3713. In this manner, any stimulus (e.g., heating or cooling) provided to the sample 3261 by the block 3710 or any mechanisms attached thereto can be provided in a substantially spatially uniform manner.

As shown in FIG. 11, the first optical member 3831 is disposed at least partially within the block 3710 such that the first optical member 3831 defines a first light path 3806 and is in optical communication with the reaction volume 3713. In this manner, a light beam (and/or an optical signal) can be conveyed between the reaction volume 3713 and a region outside of the block 3710 via the first optical member 3831. The first optical member 3831 can be any suitable structure, device and/or mechanism through which or from which a light beam can be conveyed, of the types shown and described herein. In some embodiments, the first optical member 3831 can be any suitable optical fiber to convey a light beam, such as, for example, a multi-mode fiber or a single-mode fiber.

The second optical member 3832 is disposed at least partially within the block 3710 such that the second optical member 3832 defines a second light path 3807 and is in optical communication with the reaction volume 2713. In this manner, a light beam (and/or an optical signal) can be conveyed between the reaction volume 3713 and a region outside of the block 3710 via the second optical member 3832. The second optical member 3832 can be any suitable structure, device and/or mechanism through which or from which a light beam can be conveyed, of the types shown and described herein. In some embodiments, the second optical member 3832 can be any suitable optical fiber to convey a light beam, such as, for example, a multi-mode fiber or a single-mode fiber.

As described above, the first optical member 3831 and the second optical member 3832 are coupled to the optics assembly similar to the optics assembly 2800 (not shown in FIG. 11). The optics assembly can produce one or more excitation light beams, and can detect one or more emission light beams. Thus, one or more excitation light beams can be conveyed into the reaction volume 3713 and/or the reaction container 3260, and one or more emission light beams can be received from the reaction volume 3713 and/or the portion 3261 of the reaction container 3260. More particularly, the first optical member 3831 can convey an excitation light beam from the optics assembly into the reaction volume 3713 to excite a portion of the sample 3261 contained within the reaction container 3260. Similarly, the second optical member 3832 can convey an emission light beam produced by an analyte or other target within the sample 3261 from the reaction volume 3713 to the optics assembly. In this manner, the optics assembly can monitor a reaction occurring within the reaction container 3260.

As shown in FIG. 11, the portion of first optical member 3831 and the first light path 3806 are disposed substantially within a first plane P_{XU}. The first plane P_{XY} is substantially parallel to and/or includes the longitudinal axis L_{A} of the reaction volume 3713. In other embodiments, however, the first plane P_{XY} need not be substantially parallel to and/or include the longitudinal axis L_{A} of the reaction volume 3713. The portion of second optical member 3832 and the second light path 3807 are disposed substantially within a second plane P_{YZ}. The second plane P_{YZ} is substantially parallel to and/or includes the longitudinal axis L_{A} of the reaction volume 3713. In other embodiments, however, the second plane P_{YZ} need not be substantially parallel to and/or include the longitudinal axis L_{A} of the reaction volume 3713. Moreover, as shown in FIG. 11, the first light path 3806 and the second light path 3807 define an offset angle Θ that is greater than approximately 75 degrees. More particularly, the first light path 3806 and the second light path 3807 define an offset angle Θ, when viewed in a direction substantially parallel to the longitudinal axis L_{A} of the reaction volume 3713 (i.e., that is within a plane substantially normal to the first plane P_{XY} and the second plane P_{YZ}) that is greater than approximately 75 degrees. In a similar manner, the first optical member 3831 and the second optical member 3832 define an offset angle Θ that is greater than approximately 75 degrees. This arrangement minimizes the amount of the excitation light beam that is received by the second optical member 3832 (i.e., the “detection” optical member), thereby improving the accuracy and/or sensitivity of the optical detection and/or monitoring.

In some embodiments, the portion of the instrument 3002 can produce the first light path 3806 and the second light path 3807 within the reaction volume 3713 such that the offset angle Θ is between approximately 75 degrees and approximately 105 degrees. In some embodiments, the portion of the instrument 3002 can produce the first light path 3806 and the second light path 3807 within the reaction volume 3713 such that the offset angle Θ is approximately 90 degrees.

Although the portion of the instrument 3002 is shown as producing the first light path 3806 and the second light path 3807 that are substantially parallel and that intersect in the reaction volume 3713 at a point PT, in other embodiments, the block 3713, the first optical member 3831 and/or the second optical member 3832 can be configured such that the first light path 3806 is non parallel to and/or does not intersect the second light path 3807. For example, in some embodiments, the first light path 3806 and/or the first optical member 3831 can be parallel to and offset from (i.e., skewed from) the second light path 3807 and/or the second optical member 3831. Similarly stated, in some embodiments, the first optical member 3831 and the second optical member 3832 can be spaced apart from a reference plane defined by the block 3710 by a distance Y₁ and Y₂, respectively, wherein Y₁ is different than Y₂. Thus, the position along the longitudinal axis L_{A} at which the first optical member 3831 and/or the first light path 3806 intersects the reaction volume 3713 is different from the position along the longitudinal axis L_{A} at which the second optical member 3832 and/or the second light path 3807 intersects the reaction volume 3713. In this manner, the first light path 3806 and/or the first optical member 3831 can be skewed from the second light path 3807 and/or the second optical member 3831.

In other embodiments, an angle γ₁ defined by the longitudinal axis L_{A} and the first light path 3806 and/or the first optical member 3831 can be different than an angle γ₂ defined by the longitudinal axis L_{A} and the second light path 3807 and/or the second optical member 3832 (i.e., the first light path 3806 can be non parallel to the second light path 3807). In yet other embodiments, the block 3713, the first optical member 3831 and/or the second optical member 3832 can be configured such that the first light path 3806 intersects the second light path 3807 at a location outside of the reaction volume 3713.

The distance Y₁ and the distance Y₂ can be any suitable distance such that the first optical member 3831 and the second optical member 3832 are configured to produce and/or define the first light path 3806 and the second light path 3807, respectively, in the desired portion of the reaction container 3260. For example, in some embodiments, the distance Y₁ can be such that the first optical member 3831 and/or the first light path 3806 enter and/or intersect the reaction volume 3713 at a location below the location of fill line FL of the sample 3261 when the reaction container 3260 is disposed within the block 3710. As described herein, in some embodiments an evaporation barrier including a series of beads and/or microspheres can be formed along a top surface of the sample 3261 (i.e., at the fill line FL). In this manner the excitation light beam conveyed by the first optical member 3831 will enter the sample 3261 below the fill line and/or below the evaporation barrier. This arrangement can improve the optical detection of analytes within the sample by reducing attenuation of the excitation light beam that may occur by transmitting the excitation light beam through the head space of the reaction container (i.e., the portion of the reaction container 3260 above the fill line FL that is substantially devoid of the sample 3261) and/or an evaporation barrier present along the surface of the sample 3261. In other embodiments, however, the distance Y₁ can be such that the first optical member 3831 and/or the first light path 3806 enter the reaction volume 3713 at a location above the location of fill line FL of the sample 3261 when the reaction container 3260 is disposed within the block 3710.

Similarly, in some embodiments, the distance Y₂ can be such that the second optical member 3832 and/or the second light path 3807 enter and/or intersect the reaction volume 3713 at a location below the location of fill line FL of the sample 3261 when the reaction container 3260 is disposed within the block 3710. As described herein, in some embodiments an evaporation barrier including a series of beads and/or microspheres can be formed along a top surface of the sample 3261 (i.e., at the fill line FL). In this manner the emission light beam received by the second optical member 3832 will exit the sample 3261 below the fill line FL and/or below the evaporation barrier. This arrangement can improve the optical detection of analytes within the sample by reducing attenuation of the emission light beam that may occur by receiving the emission light beam through the head space of the reaction container. In other embodiments, however, the distance Y₂ can be such that the second optical member 3832 and/or the second light path 3807 enter and/or intersect the reaction volume 3713 at a location above the location of fill line FL of the sample 3261 when the reaction container 3260 is disposed within the block 3710.

Referring again to FIGS. 6-9, as described above, the PCR sample 1261 can be prepared using lyophilized reagents, liquid reagents, or a combination of both and is disposed within and/or transferred to the PCR vial 1260 to undergo a PCR process. Within the PCR vial 1260, some amount of gaseous volume 1262 (also referred to as “head space”) can be present along with the liquid PCR sample 1261. As such, absent any mechanism for reducing evaporation (e.g., an evaporation barrier further described herein), during the PCR process a portion of the liquid PCR sample 1261 can be evaporated into the gaseous volume 1262 within the PCR vial 1260. Furthermore, some of the evaporated portion from the PCR sample 1261 can condense on a portion of the walls of the PCR vial 1260 that are not otherwise covered by the liquid PCR sample 1261. The rate or amount of evaporation of the liquid PCR sample 1261 can vary as a function of the temperature during the thermal cycling of the PCR vial 1260. Similarly, the rate or amount of condensation of evaporated portion from the PCR sample 1261 can vary as a function of the temperature during the thermal cycling of the PCR vial 1260. In some embodiments, the evaporation of the PCR sample 1260 can lead to inaccurate and/or insufficient results of a PCR process. In this manner, an evaporation barrier can be employed to reduce the amount of evaporation of a PCR sample.

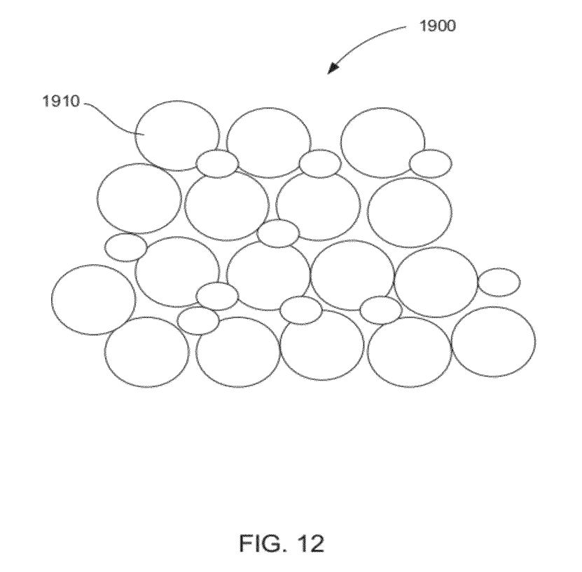

For example, FIG. 12 shows an evaporation barrier 1900 according to an embodiment that can be formed along the top surface (i.e., the fill line FL, as shown in FIG. 11). The evaporation barrier 1900 includes one or multiple layers of spherical beads 1910 (also referred to herein as microspheres 1910). More specifically, in use, the evaporation barrier 1900 can be disposed at the interface of the liquid PCR sample 1261 and the gaseous volume 1262 within PCR vial 1260 (e.g., FIGS. 7 and 9). By reducing the surface area of the liquid PCR sample 1261 from which liquid molecules can evaporate and/or increasing the path lengths for evaporation, the evaporation barrier 1900 can reduce the rate or amount of evaporation of the PCR sample 1261. Similarly stated, the evaporation barrier 1900 decreases the surface area of the liquid PCR sample 1261 that is directly exposed to the gaseous volume 1262.

In some embodiments, the beads 1910 can be constructed from any suitable material configured to minimize interference with or adverse affects on the desired reaction in the PCR vial 1260. In some embodiments, the beads 1910 can be constructed from a material that is inert to the samples, reagents, and/or any given constituent introduced to the PCR vial 1260. For example, in some embodiments, the beads 1910 are constructed from an inert polymer such as polystyrene, polyethylene, polyethene, polypropylene, neoprene, or the like. In this manner, the material used to form the beads 1910 of the evaporation barrier 1900 can be such that the beads 1910 maintain a given phase (e.g., the solid phase). Similarly stated, the material used to form the beads 1910 can be such that the beads 1910 need not move through a phase change during the thermal cycling of a PCR process.

In some embodiments, the beads 1910 can be, at least in part, constructed from a magnetite such that the beads 1910 possess paramagnetic properties. For example, in some embodiments, the beads 1910 can include a magnetic core substantially surround by polystyrene. In this manner, the beads 1910 can be aligned, transferred, moved, or otherwise configured via an applied magnetic force. Furthermore, the beads 1910 can be coated with a material such that the surface of the beads 1910 is hydrophobic to the liquid PCR sample 1261. In this manner, the liquid PCR sample 1261 will not readily wet the surface of the beads 1910, thus enhancing the performance of the evaporation barrier 1900. In some embodiments, the beads 1910 can be buoyant in the liquid PCR sample 1261, so that the beads 1910 will rise to and/or remain at the surface of the liquid PCR sample 1261. Such buoyancy of the beads 1910 in the liquid PCR sample 1261 can be a result of the material from which the beads 1910 are constructed and/or a hollow construction of the beads 1910. For example, in some embodiments, the beads 1910 can be hollow and constructed from chlorinated polypropylene, so that the beads 1910 are both buoyant and hydrophobic in a typical liquid PCR sample 1261 for amplifying isolated nucleic acid.

In some embodiments, the beads 1910 can be characterized by a size distribution that allows a close packing of the beads 1910. More specifically, as shown in FIG. 12, the beads 1910 can have multiple sizes and/or diameters. In this manner, the effectiveness of the evaporation barrier 1900 can be improved, as smaller beads 1910 can cover gaps in the surface area of the liquid PCR sample 1261 left uncovered by larger beads 1910. The particular distribution of bead sizes can depend, among other things, on the geometry and/or size of the PCR vial 1260. In some embodiments, for example, the bead diameters can be chosen to range between 100 and 500 microns for use with a 200 micro-liter PCR vial. In other embodiments, a first portion of the beads can have a first size distribution (e.g., a nominial size of 100 microns) and second portion of the beads can have a second size distribution (e.g., a nominal size of 4-6 microns).

In some embodiments, the beads and/or microspheres 1910 are disposed within a storage module, and are configured to be delivered to a portion of a cartridge during a PCR process. For example, in some embodiments, the beads 1910 are disposed with the cartridge 1001 described above in FIGS. 1-9. More specifically, in some embodiments, the beads 1910 are stored within the wash buffer module 1130b of the isolation module 1100 and are transferred to the elution chamber 1190 during a PCR process.

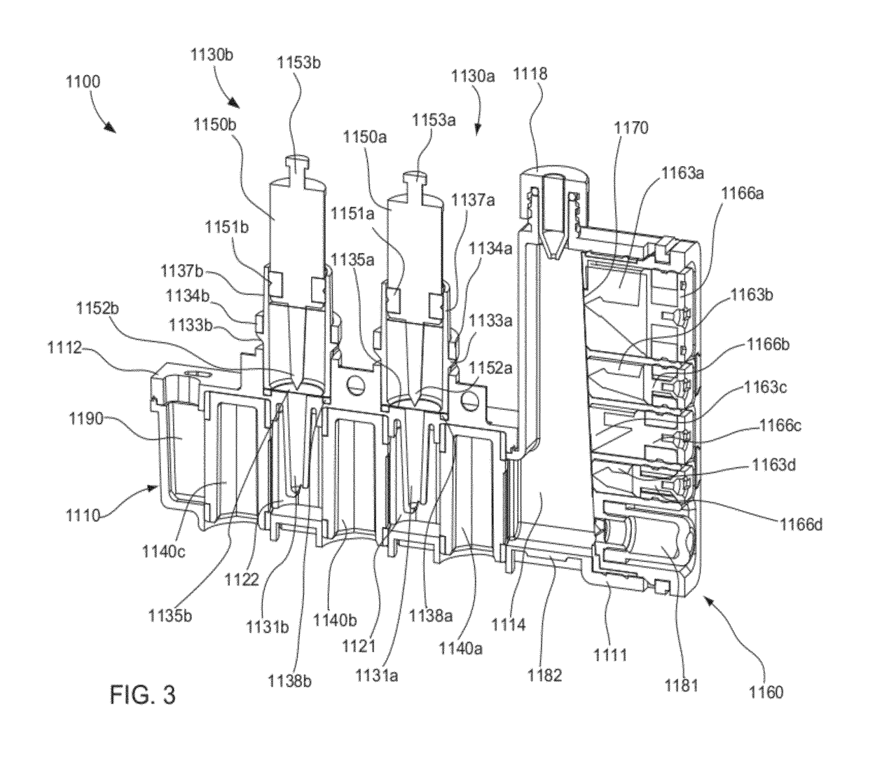

As described above with respect to FIGS. 3 and 4, the wash buffer module 1130b includes the actuator 1150b that is movably disposed within the housing 1137b. The actuator 1150b can be engaged by an instrument (e.g., the instrument 2002) such that the piercing portion 1152b pierces the puncturable member 1135b to deliver the beads 1910 to the wash chamber 1122 (as indicated by the arrow BB in FIG. 4). In this manner, the beads 1910 can be added to the PCR sample 1261 delivered to the wash chamber 1122 via the transfer assembly 1140b. Furthermore, the beads 1910 and the PCR sample 1261 can be delivered to the elution chamber 1910 as described above.

In some embodiments, the wash buffer module 1130b can be configured to contain a wash buffer (e.g., mineral oils or the like) and the beads 1910. In other embodiments, the wash buffer module 1130b need not include a wash buffer 1130b. Similarly stated, the wash buffer module 1130b can be configured to only include the beads 1910 such that the beads 1910 are fluidically isolated from a portion outside the wash buffer module 1130b until the piercing portion 1152b pierces the puncturable member 1135b. While described above as being disposed within the wash buffer module 1130b, in other embodiments, the beads 1910 can be disposed within the wash buffer module 1130a and added to the PCR sample 1261 in the wash chamber 1121. In this manner, the PCR sample 1261 and the beads 1910 can be transferred to the wash chamber 1122 and to the elution chamber 1190.

With the PCR sample 1261 and the beads 1910 disposed within the elution chamber 1190, the transfer mechanism 1235 of the PCR module 1200 (e.g., FIGS. 8 and 9) can be engaged to transfer the PCR sample 1261 or a portion of the PCR sample 1261 from the elution chamber 1190 to the PCR vial 1260 via the fluid passageway 1222. In such embodiments, the negative pressure introduced by the transfer member 1235 can further urge the beads 1910 (e.g., microspheres) to flow within the fluid passageway 1222 and into the PCR vial 1260. In some embodiments, the beads 1910 can be configured to be transferred concurrently with at least a portion of the PCR sample 1261. In other embodiments, the beads 1910 can be configured to be transferred substantially after the PCR sample.

Furthermore, in some embodiments, one or more beads 1910 can be configured to substantially seal the fluid passageway 1222 once the desired amount or distribution of beads 1910 is conveyed to the PCR vial 1260. For example, in some embodiments, the elution chamber 1190 can include a bead of substantially greater density configured to be the last bead 1910 transferred through the fluid passageway 1222. In some embodiments, the fluid passageway 1222 can include a feature and/or inner surface (e.g., a step or taper) configured to engage the bead 1910 such that the bead substantially seals the fluid passageway 1222. In other embodiments, one or more beads 1910 can be formulated to swell after being introduced into the sample within the elution chamber 1190 and/or the wash chamber 1122. In such embodiments, the bead 1910 can be configured such that the rate of expansion of the bead 1910 corresponds to a time period when the bead 1910 is disposed within the fluid passageway 1222. In this manner, the bead 1910 can expand to substantially seal the fluid passageway 1222 at any suitable location within the fluid passageway 1222. Similarly stated, the expansion of the bead 1910 can be such that the PCR vial 1260 is fluidically isolated from the elution chamber 1190 and/or a portion of the fluid passageway 1222. In other embodiments, the elution chamber 1910 can include, for example, mineral oils configured to substantially seal the fluid passageway 1222 once the PCR sample 1261 and/or the beads 1910 have been transferred to the PCR vial 1260. By sealing the fluid passageway 1222 and/or fluidically isolating portions of the cartridge from the PCR vial 1260 by blocking the fluid passageway 1222, the amount of heat and/or mass transfer of the sample during the PCR can be further reduced, thereby increasing the accuracy of the assay.

While described above as being included in the wash buffer module 1130b, in some embodiments, the beads 1910 can be disposed within the reagent module 1270a. As described above with respect to FIGS. 6 and 7, the reagent module 1270a includes the actuator 1280a that is movably disposed within the housing 1277a. The actuator 1280a can be engaged by an instrument (e.g., the instrument 2002) such that the piercing portion 1282a pierces the puncturable member 1275a to deliver the substance R1 and/or the beads 1910 to the elution chamber 1190, as described in detail above. With the beads 1910 delivered to the elution chamber 1190, the PCR sample 1261 and the beads 1910 can be transferred to the PCR vial 1260, as described above.

In some embodiments, the beads 1910 can be disposed within the reagent module 1270b. As described above with respect to FIGS. 6 and 7, the reagent module 1270b includes the actuator 1280b that is movably disposed within the housing 1277b. The actuator 1280b can be engaged by an instrument (e.g., the instrument 2002) such that the piercing portion 1282b pierces the puncturable member 1275b to deliver the substance R2 and/or the beads 1910 to the PCR vial 1260. More specifically, the movement of the actuator 1280b in the direction of the arrow DD in FIG. 7, places the substance R2 and/or the beads 1910 in fluid communication with the flow passageway 1221b. In this manner, the substance R2 and/or the beads 1910 can be transferred to the PCR vial 1260. In some embodiments, the beads 1910 can be configured to seal the flow passageway 1221b in a similar manner as described above with reference to the flow passageway 1222.

In some embodiments, the beads 1910 can be disposed within the volume 1237 of the transfer assembly or mechanism 1235. As described above, the transfer mechanism 1235 can be configured such that the movement of the actuator 1240 within the housing 1236 introduces a negative pressure within the PCR vial 1260, thereby drawing the PCR sample 1261 into the PCR vial 1260 from the elution chamber 1190. Moreover, when the transfer mechanism 1235 is actuated, the volume 1237 defined therein is placed in fluid communication with the PCR volume 1260 via the passageway 1223, as described above. Thus, in some embodiments, the beads 1910 can be added to the PCR vial 1260 via the same operation as the sample transfer operation.

In some embodiments, a PCR vial 1260 can include the constituents that form the evaporation barrier 1900 in advance by pre-filling the PCR vial 1260 with a particular type, distribution, and quantity of beads 1910 before use (e.g., prior to receiving the PCR sample 1261). In some embodiments, the PCR vial 1260 pre-filled with beads 1910 can be further pre-filled with lyophilized reagents for a particular type of desired reaction. In use, liquid samples and/or reagents can be transferred into the PCR vial 1260 that has been pre-filled with beads 1910, such that the buoyancy of the beads 1910 facilitates the formation of the evaporation barrier 1900 on the top surface of the liquid PCR sample 1261 (e.g., at the fill level FL shown and described above with respect to FIG. 12). Moreover, with the evaporation barrier 1900 disposed substantially at the fill level FL, the evaporation barrier 1900 and/or any of the beads 1910 comprising the evaporation barrier 1900 are disposed such that the optical detection is substantially free from interference from the evaporation barrier 1900, as described above with reference to the instrument 3002.

In other embodiments, the beads 1910 can be formulated to bind with a specific analyte to facilitate optical detection. In this manner, the beads 1910 can form an evaporation barrier (as described herein) during the PCR, and can facilitate optical detection of the PCR products. For example, in some embodiments, a portion of the beads 1910 can be treated with a DNA oligonucleotide that specifically hybridizes to a DNA target of interest. In such embodiments, the portion of the beads 1910 can include a non-fluorescent quencher at the 3′ end and a fluorophore at the 5′ end. Moreover, the portion of the beads 1910 can also include multiple different types of beads, each type having a different binding capacity and/or that is configured to produce a different optical signal. For example, in some embodiments, the beads 1910 can be constructed from polystyrene and magnetite. The beads 1910 can include, for example, a first set that is hybridized and/or formulated to have a first binding capacity (e.g., the capability to bind to a single target molecule) and a second set that is hybridized and/or formulated to have a second binding capacity (e.g., the capability to bind to two target molecules). Moreover, the different bead types can each have a different dye or marker such that the different types can be differentiated during optical detection. In some embodiments, such as those wherein the evaporation barrier 1900 includes multiple sized beads 1910, the detection beads can be substantially smaller than adjacent non-detection beads. For example, in some embodiments, the detection beads can be approximately 6 microns. In other embodiments, the detection beads can be approximately 2 microns, 3 microns, 4 microns, 5 microns, 7 microns, 8 microns, 9 microns, and/or 10 microns. In this manner, the detection beads can cover and/or fill the gaps in the surface area of the liquid PCR sample 1261 left uncovered by larger beads 1910 (e.g., those beads having a size of 100 to 500 microns), and are also formulated to bind to a specific target.

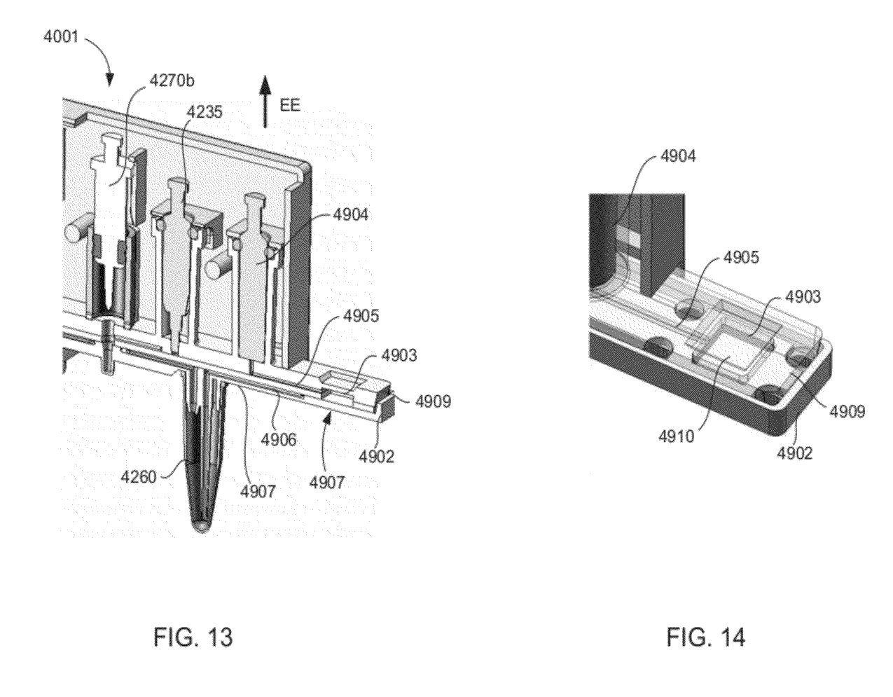

In some embodiments, the portion of beads 1910 formulated to bind to target analytes (e.g., detection beads) can be transferred from a PCR vial to a secondary detection chamber for optical detection. For example, FIGS. 13 and 14 illustrate a cartridge 4001 having a reaction volume that is distinct from (e.g., at a different spatial location from) the detection volume. In some embodiments, the cartridge 4001 can be used to process the sample and conduct PCR product labeling as described above. The cartridge 4001 can be substantially similar to the cartridge 1001 described above, and is therefore not described in detail herein. For example, the cartridge 4001 can include any suitable reagent modules, for example, the reagent modules 4270b, which is similar to the reagent module 1270b shown and described above. The cartridge 4001 includes a transfer mechanism 4235 and a PCR vial 4260 which are similar to the transfer mechanism 1235 and the PCR vial 1260 shown and described above. In this manner, the cartridge 4001 can be manipulated in a similar manner as described herein (e.g., via the instrument 2002).

As described above, the cartridge 4001 can be configured to include a set of beads configured to form an evaporation barrier along the surface of the sample (e.g., within the PCR vial 4260) during a PCR process. Furthermore, a portion of the beads (not shown in FIGS. 13 and 14) is formulated to bind to specific target analytes. In some embodiments, the beads can be disposed within the reagent module 4270b and transferred to the PCR vial 4260, as described above with respect to the reagent module 1270b. In other embodiments, the beads can be disposed within the transfer mechanism 4235 and transferred to the PCR vial 4260, as described above with respect to the transfer mechanism 1235. In still other embodiments, the PCR vial 4260 can be prefilled with the beads prior to receiving a PCR sample. In yet other embodiments, the beads can be disposed in any other suitable chamber and transferred to the PCR vial 4260 in any suitable manner.

The cartridge 4001 differs from the cartridge 1001, however, in that the cartridge 4001 includes a flow cell portion 4903 within which detection and/or analysis can occur. Expanding further, the cartridge 4001 includes a second transfer mechanism 4904 and an extension or end portion 4902 configured to extend from a portion of the cartridge 4001 such that a flow cell portion 4903 of the cartridge 4001 can be engaged by an optical detection system (not shown). Similarly stated, as described below, the flow cell portion 4903 is included within the protruding end portion 4902, thereby providing substantially unobstructed access to a detection volume 4910 of the flow cell portion 4903.

As shown in FIG. 13, the cartridge 4001 includes a first layer (or base) 4907 and a second layer 4909. The cartridge 4001 (and/or the first layer 4907 and the second layer 4909) defines a first flow path 4906 and a second flow path 4905. More specifically, the first flow path 4906 is in fluid communication with the PCR vial 4260 (i.e., a reaction volume) and the detection volume 4910. Thus, a sample (including any portion the beads contained therein) can be conveyed from the reaction volume to the detection volume 4910 via the first flow path 4906. The second flow path 4905 is in fluid communication with the second transfer mechanism 4904 and the detection volume 4910 of the flow cell portion 4903. In this manner, when the second transfer mechanism 4904 is actuated, a portion of the sample (e.g., a portion of the sample bound to the detection beads) within the PCR vial 4260 can be conveyed into the flow cell portion 4903 and/or the detection volume 4910.

As shown in FIG. 13, the first flow path 4906 and/or the second flow path 4905 define a multi-directional flow path. In this manner, when the second transfer mechanism 4904 is actuated, the a first portion of the detection beads (and the target analyte bound to the detection beads) flows within the first flow path 4906 in a first direction and a second portion of the detection beads (and/or waste product) flows within the second flow path 4905 in a second direction, opposite the first. In this manner, the distance the extension 4902 extends beyond the portion of the cartridge 4001 can controlled to accommodate the detection equipment of the instrument within which the cartridge 4001 is disposed (not shown herein). In some embodiments, the extension 4902 can be configured to extend a desired distance from the portion of the cartridge 4001 such that the extension 4902 can be interfaced with an optical module or the like.

As described above, the second transfer mechanism 4904 moves the labeled product (e.g., the sample and the beads) from the PCR vial 4260 to the flow cell portion 4903, which is integrated in the cartridge 4001. In particular, the second transfer mechanism 4904 includes a plunger that is moved upward, as shown by the arrow EE in FIG. 13, which produces a vacuum within the detection volume 4910 of the flow cell portion 4903. Moreover, the movement of the plunger opens a volume within the second transfer mechanism 4904 within which a portion of the sample and/or waste products can flow after passing through the flow cell portion 4903. In use, after a portion of labeled PCR products have been conveyed into the detection volume 4910, the PCR products can be detected by any suitable mechanism.

For example, in some embodiments, as described above, the PCR products are labeled with and/or attached to magnetic beads. Moreover, in some embodiments, the magnetic beads are a subset of beads from the beads that form an evaporation barrier. The beads can include a series of hybridized detection beads of the type described above. In such embodiments, detection can include applying a magnetic field to a first surface that defines the detection volume 4910 (e.g., a portion of the first layer 4907). In this manner, the magnetic particles and sample adhered and/or bound thereto can be maintained against a surface (either the first surface or layer 4907 or an opposing second surface, e.g., the second layer 4909). While the particles are maintained in position against the surface, the sample can be excited by one or more light sources having any desired wavelength. An optical detection system (e.g., a CCD camera, photodiode or the like) can then measure the light emitted from the sample, which can be used to produce a map of the sample resident within the detection volume 4910. The optics assembly can include any of the components as described herein. The optics assembly can include, for example, a magnet, a series of LEDs, a CCD camera or the like. The architecture of the optics module 2800, as described herein, can be modified in order to allow for detection of the PCR product in the flow cell 4903.

In some embodiments, for example, the sample and beads can be excited sequentially by multiple different light sources, each having a different wavelength. This can result in different light emissions produced by the samples and/or beads, and can allow for quantization and/or accurate characterization of the sample. In such embodiments, the detection beads can be dyed with various colors such that detection of various analytes can be performed.

In some embodiments, the cartridge 4001 can include the hybridized detection beads within the second transfer mechanism 4904. Thus, in use, when the plunger of the second transfer mechanism 4904 is moved upward, as shown by the arrow EE in FIG. 13, the sample is drawn into the second transfer mechanism 4904 and is mixed with the beads stored therein. The plunger can then be moved in the opposite direction to convey the sample and the beads in the detection volume 4910 for optical detection. In other embodiments, the beads can be included in the reagent module 4270b, which is sealed with a puncturable member, as described herein. In this manner, the beads and the solution within which they are contained can be packaged separately from the construction of the cartridge 4001, and can be later coupled to the cartridge as described herein.

The flow cell 4903 is designed so that the labeled product accumulates in the read area 4910 while still allowing for flow to occur (e.g., through the first flow path 4905 and the second flow path 4906). Similarly stated, the arrangement presented above allows for waste and/or return flow to accumulate within the second transfer mechanism 4904, the PCR vial 4260 or any other suitable chamber within the cartridge 4001. In some embodiments, the flow cell portion 4903 can include a flow structure (e.g., an obstruction, a series of structures that produce a tortuous path or the like) that limits and/or controls the passage of the magnetic particles through the detection volume. In this manner, the flow cell portion 4903 can be configured for use with a detection system based on flow cytometry principles.

FIG. 14 shows a graph comparing the performance of the evaporation barriers described herein with evaporation barriers formed from mineral oils. In particular, the graph in FIG. 14 shows the number of temperature cycles executed during a PCR process on the x-axis, and the level of amplification signal, such as florescence, on the y-axis. In a PCR process, the amplification signal can indicate the level of concentration of amplified DNA within the sample. Thus, the point at which the amplification signal rises dramatically indicates that PCR process has progressed to an exponential amplification stage.

As shown in FIG. 14, two PCR processes were conducted using mineral oil as a control. Three PCR processes were conducted using an evaporation barrier including beads. In particular, the PCR vials included five mg of hollow chlorinated polypropylene beads with diameters between 100 and 500 microns. In both groups (i.e., either mineral oil or beads as the evaporation barrier) FIG. 14 shows that the PCR processes enter the exponential amplification stage after substantially the same number of thermal cycles. As such, the experiment shows that the use of beads is an effective evaporation barrier and can be a viable alternative to mineral oils in the PCR process.

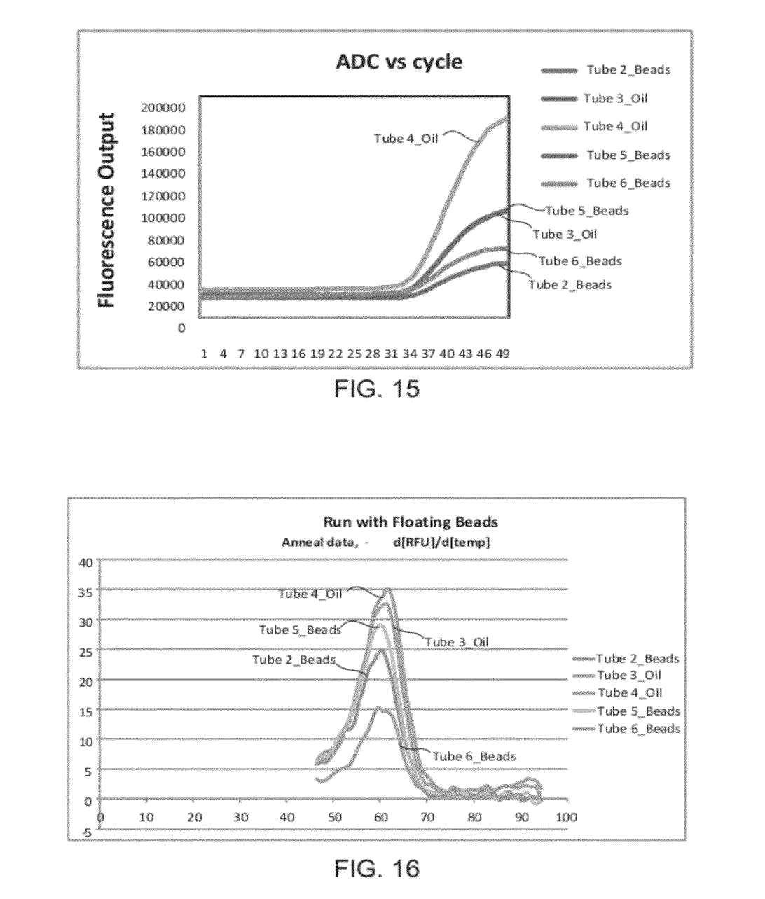

FIG. 15 shows a graph comparing the anneal temperatures of the amplified samples produced by the PCR processes shown in FIG. 14. The graph in FIG. 15 shows temperature on the x-axis, and the Relative Florescence Unit on the y-axis, with the peak of the curves occurring at the anneal temperature of the reactions. Since the peaks of the curves in FIG. 15 occur at substantially similar temperatures, FIG. 15 shows that all five experiments had substantially similar anneal temperatures. The anneal temperatures are correlated to the melting temperatures of DNA primer samples used in the reaction, thus FIG. 15 confirms that all five experiments used DNA primer samples that have substantially similar melting temperatures.

A DNA primer is a strand of nucleic acid that can serve as a starting point in a PCR process. The particular sequence of a DNA primer can correspond to a particular melting temperature. Because the experiments underlying FIGS. 14 and 15 used the same set of DNA primers for each experiment, the confirmation in FIG. 15 that each of the five experiments had substantially similar anneal temperatures and melting temperatures verifies that the use of beads is an effective evaporation barrier. Similarly stated, these test results demonstrate that using beads as an evaporation barrier provides substantially similar PCR results as the use of mineral oils.

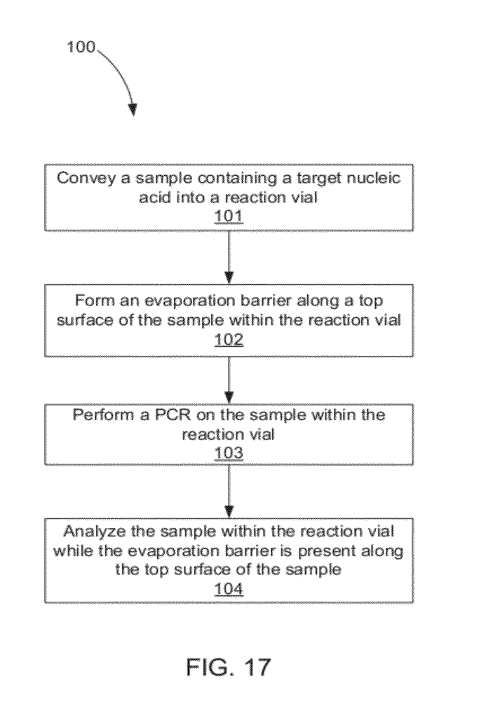

FIG. 17 is a flowchart illustrating a method 100 for establishing an evaporation barrier during a PCR process, according to an embodiment. The method 100 can be performed in any of the apparatus described herein. For example, in some embodiments, the method 100 can be performed in the cassette 1001 that is manipulated via the instrument 2002. The method 100 includes conveying a sample containing a target nucleic acid into a reaction vial at 101. The reaction vial can be any suitable reaction vial such as, for example, the PCR vial 1260 described above with respect to FIGS. 1-9. In such embodiments, the reaction vial is configured to include multiple particles configured to form an evaporation barrier. More specifically, in some embodiments, the particles can be beads or microspheres of the types shown and described herein. For example, in some embodiments, the particles can be the beads 1910 configured to form the evaporation barrier 1900, described above with respect to FIG. 12. Thus, the particles can be formulated to be inert and/or hydrophobic to the sample. In some embodiments, the reaction vial can be configured to be pre-filled with the particles, as described above. In other embodiments, the particles can be transferred to the reaction vial via, for example, a transfer mechanism included in a storage module. In such embodiments, the particles can be conveyed to the reaction vial before, after, and/or concurrently with the sample.

With the sample conveyed to the reaction vial, the method 100 includes forming an evaporation barrier along the top surface of the sample within the reaction vial at 102. For example, as described above the particles (e.g., beads) can be configured to be buoyant when disposed in or on the sample. In this manner, the particles are can be disposed on a top surface of the sample to substantially form the evaporation barrier. With the particles disposed on the top surface of the sample, the method 100 includes performing a PCR process on the sample within the reaction vial at 103. For example, in some embodiments, the PCR process includes thermal-cycling the reaction vial and the sample disposed therein. In this manner, the particles can form the evaporation barrier that substantially reduces the amount the sample evaporates during thermal-cycling.

The method further includes analyzing the sample within the reaction vial while the evaporation barrier is present along the top surface of the sample at 104. For example, in some embodiments, the analyzing includes optically analyzing a fluorescence output of at least a portion of the sample. In some embodiments, the analyzing can be performed during the PCR process. In other embodiments, the analyzing can be performed after the PCR process. Furthermore, the evaporation barrier can be disposed relative to the sample and/or the optical detection assembly (e.g., the optical detection assembly 2800 included in the instrument 2002 described herein) such that the evaporation barrier does not substantially interfere with the optical detection. In some embodiments, such as for example those described above with respect to FIG. 11, an optical detection assembly can produce a light beam such that the path of the light beam is substantially below the evaporation barrier (e.g., substantially disposed at the fill level FL).

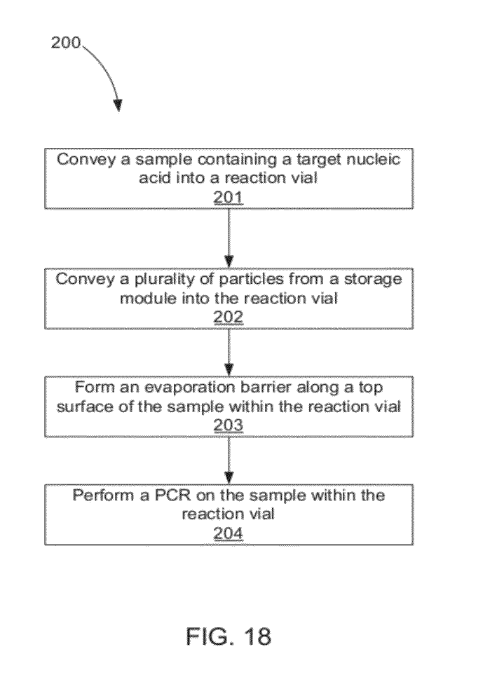

FIG. 18 is a flowchart illustrating a method 200 for establishing an evaporation barrier during a PCR process, according to an embodiment. The method 200 can be performed in any of the apparatus described herein. For example, in some embodiments, the method 100 can be performed in the cassette 1001 via the instrument 2002. In this manner, the method 200 includes conveying a sample containing a target nucleic acid into a reaction vial at 201. For example, in some embodiments, the sample, can be conveyed from a module or chamber via a flow passageway (e.g., from the elution chamber 1190 via the flow passageway 1222 included in the cassette 1001). The reaction vial can be any suitable reaction vial such as, for example, the PCR vial 1260 described above with respect to FIGS. 1-9.

The method 200 further includes conveying multiple particles from a storage module into the reaction vial at 202. The storage module can be any suitable storage module described herein. For example, in some embodiments, the storage module can be a wash buffer module (e.g., the wash buffer module 1130a or 1130b), an elution chamber (e.g., the elution chamber 1190), a reagent module (e.g., the reagent module 1270a or 1270b), a transfer mechanism (e.g., the transfer mechanism 1235), and/or any other suitable storage module. Furthermore, the storage module can include a portion configured to be actuated such that a volume defined by the storage module is placed in fluid communication with the reaction vial. In some embodiments, the particles can be beads or microspheres. For example, in some embodiments, the particles can be the beads 1910 configured to form the evaporation barrier 1900, described above with respect to FIG. 12. In this manner, the particles can be configured to be buoyant, inert, and hydrophobic with respect to the sample.

With the sample and the particles disposed within the reaction vial, the method 100 includes forming an evaporation barrier along the top surface of the sample within the reaction vial at 203. For example, as described above the particles (e.g., beads) can be configured to be buoyant when disposed in or on the sample. In this manner, the particles are can be disposed on a top surface of the sample to substantially form the evaporation barrier. With the particles disposed on the top surface of the sample, the method 200 includes performing a PCR process on the sample within the reaction vial at 204. For example, in some embodiments, the PCR process includes thermal-cycling the reaction vial and the sample disposed therein. In this manner, the particles can form the evaporation barrier such that the evaporation barrier substantially reduces the amount the sample evaporates during thermal-cycling. Furthermore, the particles can be configured to substantially remain in the solid phase during the thermal-cycling of the PCR process.

In some embodiments, a portion of the particles that form the evaporation barrier can be configured to bind to a target nucleic acid within the sample. For example, in some embodiments, the portion of the particles can be formulated to be detection particles, as described above. In such embodiments, the target nucleic acid can bind to the detection particles to enhance optical detection. In some embodiments, the detection particles and the bound target nucleic acid can be transferred from the reaction vial to any suitable analysis module. For example, in some embodiments, the analysis module can be a flow cell (e.g., the flow cell 4903 described above with respect to FIGS. 13 and 14). In this manner, the detection particles can be configured to both facilitate the formation of the evaporation barrier and facilitate the optical detection of the analyte.

While various embodiments have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above.

For example, although the evaporation barrier is shown and described above as including a series of beads having a substantially spherical shape (e.g., microspheres), in other embodiments, an evaporation barrier can include a series of particles having any suitable shape or shapes. For example, in some embodiments, an evaporation barrier can include a series of beads having a substantially disk-like shape.

Although the evaporation barrier is shown and described above as being used in a PCR vial, in other embodiments an evaporation barrier of the types shown and described herein can be used in any suitable PCR vial.

Although various embodiments have been described as having particular features and/or combinations of components, other embodiments are possible having a combination of any features and/or components from any of embodiments where appropriate.

What is claimed:

1. An apparatus, comprising:
a sample isolation module defining a chamber configured to contain a sample;
a storage module including a transfer mechanism and defining a volume, the volume containing a plurality of particles formulated to be buoyant when disposed in the sample, the storage module configured to be coupled to the sample isolation module such that the volume can be selectively placed in fluid communication with the chamber, the transfer mechanism configured to transfer the plurality of particles from the volume to the chamber when the transfer mechanism is actuated; and
a reaction module including a transfer mechanism and defining a reaction chamber, a portion of the reaction module being disposed within the chamber of the sample isolation module when the reaction module is coupled to the sample isolation module such that the reaction chamber is fluid communication with the chamber of the sample isolation module, the transfer mechanism of the reaction module configured to transfer the sample and the plurality of particles from the chamber to the reaction chamber when the transfer mechanism of the reaction module is actuated.

2. The apparatus of claim 1, wherein:
a portion of the plurality of particles is formulated to bind to a target analyte within the sample.

3. The apparatus of claim 1, wherein:
a portion of the plurality of particles is treated with a dye formulated to produce light emission when excited.

4. The apparatus of claim 1, wherein each particle from the plurality of particles is formulated to include magnetite.

5. The apparatus of claim 1, wherein the volume contains an oil-based liquid.

6. The apparatus of claim 1, wherein a first portion of the plurality of particles has a first size distribution and a second portion of the plurality of particles has a second size distribution different than the first size distribution.

7. An apparatus, comprising:
a storage module including a first transfer mechanism and defining a volume, the volume containing a plurality of particles formulated to be buoyant when disposed in a sample, the first transfer mechanism configured to expel the plurality of particles from the volume when the first transfer mechanism is actuated; and
a reaction module including a second transfer mechanism and defining a reaction chamber, a portion of the reaction module being coupled to a sample isolation module such that the reaction chamber is fluid communication with a sample isolation chamber, the second transfer mechanism configured to transfer the sample from the sample isolation module to the reaction chamber when the second transfer mechanism is actuated,
the storage module configured to be coupled to the reaction module such that the volume can be selectively placed in fluid communication with the reaction chamber, the first transfer mechanism configured to transfer the plurality of particles from the volume to the reaction chamber when the first transfer mechanism is actuated.

8. The apparatus of claim 7, wherein the first transfer mechanism is configured to transfer the plurality of particles from the volume to the reaction chamber while maintaining the plurality of particles in isolation from a region outside of the storage module and the reaction module.

9. The apparatus of claim 7, wherein the reaction module and the storage module collectively define a flow path through which the plurality of particles is conveyed from the volume to the reaction chamber when the first transfer mechanism is actuated, the flow path being fluidically isolated from a region outside of the storage module and the reaction module.

10. The apparatus of claim 7, wherein:
a portion of the plurality of particles is formulated to bind to a target analyte within the sample.

11. A method, comprising:
conveying a sample containing a target nucleic acid into a reaction vial, the reaction vial containing a plurality of particles;
forming an evaporation barrier along a top surface of the sample within the reaction vial, the evaporation barrier including the plurality of particles;
performing a polymerase chain reaction (PCR) on the sample within the reaction vial; and
analyzing, after the performing, the sample within the reaction vial while the evaporation barrier is present along the top surface of the sample.

12. The method of claim 11, wherein the analyzing includes transmitting a light beam into the sample via a first light path that includes a portion of a wall of the reaction vial, the portion being located below the evaporation barrier.

13. The method of claim 11, wherein the plurality of particles is formulated to be buoyant when disposed in the sample.

14. The method of claim 11, wherein each particle from the plurality of particles is formulated to be inert and hydrophobic.

15. The method of claim 11, wherein the plurality of particles is formulated be in a substantially solid phase during the performing the polymerase chain reaction.

16. The method of claim 11, further comprising:
conveying the plurality of particles from a storage module into the reaction vial.

17. The method of claim 11, further comprising:
actuating a transfer mechanism of a storage module, the storage module defining a volume containing the plurality of particles, such that the plurality of particles is conveyed into the reaction vial.

18. A method, comprising:
conveying a sample containing a target nucleic acid into a reaction vial;
conveying a plurality of particles from a storage module into the reaction vial;
forming an evaporation barrier along a top surface of the sample within the reaction vial, the evaporation barrier including the plurality of particles; and
performing a polymerase chain reaction (PCR) on the sample within the reaction vial.

19. The method of claim 18, wherein the conveying the plurality of particles includes actuating a transfer mechanism of the storage module to place the storage module in fluid communication with the reaction vial.

20. The method of claim 18, wherein the conveying the sample includes actuating a transfer mechanism to convey the sample from a sample isolation module to the reaction vial.

21. The method of claim 18, wherein the plurality of particles is formulated to be buoyant when disposed in the sample.

22. The method of claim 18, wherein the plurality of particles is formulated be in a substantially solid phase during the performing the polymerase chain reaction.

23. The method of claim 18, further comprising:
binding a target nucleic acid within the sample to a portion of the plurality of particles, the portion of the plurality of particles formulated to produce a light emission when excited by an excitation light source.

24. The method of claim 23, further comprising:
conveying the sample and the plurality of particles to an analysis module.
